(19) United States
(12) Patent Application Publication
(10) Pub. No.: US 2025/0118418 A1
(43) Pub. Date: Apr. 10, 2025

(54) SYSTEM AND METHOD OF USING PERSONALIZED SONIFICATION MODELS TO COMMUNICATE HEALTH INFORMATION

(71) Applicant: UNIVERSITY OF VIRGINIA PATENT FOUNDATION, Charlottesville, VA (US)

(72) Inventors: Afsaneh Doryab, Charlottesville, VA (US); Matthew Clark, Charlottesville, VA (US)

(21) Appl. No.: 18/908,495

(22) Filed: Oct. 7, 2024

Related U.S. Application Data
(60) Provisional application No. 63/542,727, filed on Oct. 5, 2023.

(51) Int. Cl. G16H 20/70; G16H 40/67

(52) U.S. Cl. CPC G16H 20/70; G16H 40/67

(57) ABSTRACT

Systems and methods of generating music that encodes personalized information implement and/or include generating a personalized sonification model based on music modeling data pertaining to a user; receiving physiological data pertaining to the user, wherein the physiological data is related to at least one of a physical wellness or a behavioral wellness of the user; generating a melody, wherein the melody encodes a wellness information or modification based on the personalized sonification model and the physiological data; and providing the melody to the user to convey the wellness information or modification.

### CROSS-REFERENCE TO RELATED APPLICATIONS

This application claims priority to and the benefit of U.S. Provisional Application No. 63/542,727, filed Oct. 5, 2023 and titled “System and Method of Using Personalized Sonification Models to Communicate Health Information,” the entire contents of which are herein incorporated by reference for all purposes.

### STATEMENT REGARDING FEDERALLY SPONSORED RESEARCH OR DEVELOPMENT

This invention was made with government support under U.S. Pat. No. 1,816,687 awarded by the National Science Foundation. The government has certain rights in the invention.

### TECHNICAL FIELD

This disclosure relates generally to sonification and, in particular, to systems and methods for communicating health or other information using personalized sonification models.

### BACKGROUND

Personalized wellness technology aims to create distinctive yet individualized and efficient means of conveying wellness-related information. Comparative approaches offer insights into wellness using tangible mediums, such as illuminating elements, adaptive textiles, or garments that change colors. Conversely, alternative comparative methods harness existing user devices to present wellness data through ambient displays or gamification techniques, all geared towards promoting healthier lifestyles. It is important to note that, although these delivery methods may have some effectiveness, they predominantly rely on the user's visual perception.

Research has suggested that music has the potential to enhance user wellness significantly. Music therapy is an approach for addressing complex psychological conditions like depression and anxiety and alleviating symptoms of challenging diseases such as Parkinson's disease, multiple sclerosis, and even cancer. In addition to traditional music therapy, sonification technology has been used to translate real-time data into auditory cues. Sonification converts data into acoustic signals and has been used in various wellbeing contexts such as enhancing mindfulness and alleviating pain in therapeutic settings. Broader acoustic signals are frequently used to alter behavior, such as an alarm clock waking a user up. However, these interventions are typically designed for immediate relief and rely on real-time, relatively straightforward data. There exists a need for systems, methods, and media that can promote healthier lifestyles by conveying wellness information through music.

### SUMMARY

The present disclosure addresses needs in the field by presenting systems, methods, and computer readable media for, among other things, one or more of the following: a) a musical feedback system converts biobehavioral data collected from personal devices to personalized musical melodies to convey the general health status, b) using sonification to convey users' wellness levels through music, c) using sonification in delivering and communicating health and wellness status on personal devices, d) use of sonification for delivering and communicating personal health information, and e) using personalized sonification models to communicate health information and/or modify behavioral parameters.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

According to one aspect of the present disclosure, a method of generating music that encodes personalized information is provided. The method comprises generating a personalized sonification model based on music modeling data pertaining to a user; receiving physiological data pertaining to the user, wherein the physiological data is related to at least one of a physical wellness or a behavioral wellness of the user; generating a melody, wherein the melody encodes a wellness information or modification based on the personalized sonification model and the physiological data; and providing the melody to the user to convey the wellness information or modification.

According to another aspect of the present disclosure, a system for generating music that encodes personalized information. The system comprises at least one processor; and a memory operatively connected to the at least one processor and storing instructions that, when executed by the at least one processor, cause the system to perform operations comprising: generating a personalized sonification model based on music modeling data pertaining to a user, receiving physiological data pertaining to the user, wherein the physiological data is related to at least one of a physical wellness or a behavioral wellness of the user, generating a melody, wherein the melody encodes a wellness information or modification based on the personalized sonification model and the physiological data, and providing the melody to the user to convey the wellness information or modification.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example approach for converting physiological data into sonified wellness information according to various aspects of the present disclosure.

FIG. 2A illustrates example software used to create, modify, and export compositions according to various aspects of the present disclosure.

FIG. 2B illustrates example software used to create, modify, and export compositions according to various aspects of the present disclosure.

FIG. 3 illustrates an example melody according to various aspects of the present disclosure.

FIG. 4A illustrates an example interface according to various aspects of the present disclosure.

FIG. 4B illustrates an example interface according to various aspects of the present disclosure.

FIG. 5 illustrates an example melody according to various aspects of the present disclosure.

FIG. 6A illustrates an example interface according to various aspects of the present disclosure.

FIG. 6B illustrates an example interface according to various aspects of the present disclosure.

FIG. 7 illustrates an example melody according to various aspects of the present disclosure.

FIG. 8 illustrates example musical demographics according to various aspects of the present disclosure.

FIG. 9 illustrates example perceived healthiness of music features according to various aspects of the present disclosure.

FIG. 10 illustrates example perceived healthiness of music features according to various aspects of the present disclosure.

FIG. 11 illustrates example composition data according to various aspects of the present disclosure.

FIG. 12A illustrates example study results according to various aspects of the present disclosure.

FIG. 12B illustrates example study results according to various aspects of the present disclosure.

FIG. 13 illustrates example composition data according to various aspects of the present disclosure.

FIG. 14 illustrates example composition data according to various aspects of the present disclosure.

FIG. 15 illustrates example melodies according to various aspects of the present disclosure.

FIG. 16 illustrates an example quantitative analysis approach according to various aspects of the present disclosure.

FIG. 17A illustrates example study results according to various aspects of the present disclosure.

FIG. 17B illustrates example study results according to various aspects of the present disclosure.

FIG. 18 illustrates example study results according to various aspects of the present disclosure.

FIG. 19 illustrates example study results according to various aspects of the present disclosure.

FIG. 20 illustrates example chord progressions according to various aspects of the present disclosure.

FIG. 21 illustrates an example software used to create chord progressions according to various aspects of the present disclosure.

FIG. 22A illustrates example study results according to various aspects of the present disclosure.

FIG. 22B illustrates example study results according to various aspects of the present disclosure.

FIG. 23 illustrates an example machine according to various aspects of the present disclosure.

FIG. 24 illustrates an example method according to various aspects of the present disclosure.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various aspects of the present disclosure. However, it will be apparent to those skilled in the art that the various features, concepts, and aspects described herein may be implemented and practiced without these specific details.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or with any machine/system/computer/processor. Moreover, the various components may be in communication via wireless and/or hardwire or other desirable and available communication means, systems, and hardware. Moreover, various components and modules may be substituted with other modules or components that provide similar functions.

It should be appreciated that the device and related components discussed herein may take on all shapes along the entire continual geometric spectrum of manipulation of x, y, and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility, and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. For example, the devices, systems, apparatuses, modules, compositions, articles of manufacture, materials, computer program products, non-transitory computer readable medium, and methods of various embodiments of the invention disclosed herein may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section): A. U.S. Utility patent application Ser. No. 17/398,097, entitled “System, Method and Computer Readable Medium for Modeling Biobehavioral Rhythms from Mobile and Wearable Data Streams”, filed Aug. 10, 2021, and published on Feb. 17, 2022, as U.S. Publication No. 2022/0051798 A1.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, 5, and so on). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, 2-4, and so on). It is also to be understood that all numbers and fractions thereof are presumed to be modified by the term “about.”

It is also to be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as, e.g., “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g., “one or the other but not both”) when preceded by terms of exclusivity, such as, e.g., “either,” “one of,” “only one of,” or “exactly one of.”

The present disclosure includes a description of various methods. For any methods described, regardless of whether the method is described in conjunction with a flow diagram, it should be understood that unless otherwise specified or required by context, any explicit or implicit ordering of steps performed in the execution of a method does not necessarily imply that those steps must be performed in the order presented, but instead the steps may be performed in a different order and/or in parallel.

The present disclosure may be implemented on or with the use of computing devices including control units, processors, and/or memory elements in some examples. As used herein, a “control unit” may be any computing device configured to send and/or receive information (e.g., including instructions) to/from various systems and/or devices. A control unit may comprise processing circuitry configured to execute operating routine(s) stored in a memory. The control unit may comprise, for example, a processor, microcontroller, application-specific integrated circuit (ASIC), field-programmable gate array (FPGA), and the like, any other digital and/or analog components, as well as combinations of the foregoing, and may further comprise inputs and outputs for processing control instructions, control signals, drive signals, power signals, sensor signals, and the like. All such computing devices and environments are intended to fall within the meaning of the term “controller,” “control unit,” “processor,” or “processing circuitry” as used herein unless a different meaning is explicitly provided or otherwise clear from the context. The term “control unit” is not limited to a single device with a single processor, but may encompass multiple devices (e.g., computers) linked in a system, devices with multiple processors, special purpose devices, devices with various peripherals and input and output devices, software acting as a computer or server, and combinations of the above. In some implementations, the control unit may be configured to implement cloud processing, for example by invoking a remote processor.

Moreover, as used herein, the term “processor” may include one or more individual electronic processors, each of which may include one or more processing cores, and/or one or more programmable hardware elements. The processor may be or include any type of electronic processing device, including but not limited to central processing units (CPUs), graphics processing units (GPUS), ASICs, FPGAs, microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

As used herein, the term “memory” may be any storage medium, including a non-volatile medium, e.g., a magnetic media or hard disk, optical storage, or flash memory, including read-only memory (ROM), electrically programmable ROM (EPROM), electrically erasable programmable ROM (EEPROM); a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, a DVD-ROM, a Blu-ray disc, or floppy disks, on which programs may be stored and/or data communications may be buffered. The term “memory” may also include other types of memory or combinations thereof. For the avoidance of doubt, cloud storage is contemplated in the definition of memory.

Additional descriptions of aspects of the present disclosure will now be provided with reference to the accompanying drawings. The drawings form a part hereof and show, by way of illustration, specific embodiments or examples.

People can discern feelings of wellness through simple musical melodies. In one study, for example, participants assessed the perceived healthiness of customized musical compositions. Although this research offers evidence regarding music's ability to convey information about wellbeing, there remains a need to explore how music could serve as a feedback mechanism for individuals to gauge their personal health status based on daily behavioral data.

The present disclosure provides steps toward this goal by exploring the feasibility of using musical feedback to convey one's daily physical activity status. Musical feedback is defined as the incorporation of an individual's data into a song. In support of the present disclosure, a 76-day within-subject study was conducted wherein participants were tasked to share their Fitbit data and fill out daily surveys that offered musical or textual feedback on their daily step count. A system in accordance with the present disclosure created tailored models based on each participant's initial perception of musical wellness, infusing wellness indicators within musical tunes to help participants evaluate their level of physical activity. The step count recorded for each participant after getting their daily feedback was evaluated to measure how their interpretation of the musical feedback aligns with the intended message. Additionally, a qualitative assessment of participants' insights on receiving feedback through music was provided.

The analysis follows HCI guidelines for evaluating emerging behavior change strategies, focusing on understanding how the approach works in practice rather than solely demonstrating efficacy. Based on the findings, it can be seen that the musical feedback systems set forth herein can promote physical activity, especially compared to text-based approaches. While personalization helps to communicate, basic musical models have the capacity to convey wellness information effectively, thus reducing the necessity for excessive customization. In these systems, as users become more familiar with musical feedback, their understanding and interpretation of the melodies may be refined. Yet, this learning period can potentially be reduced if systems are mindful of how a user's current context might influence their interpretation of the music. Finally, modified systems may consider the pervasiveness of music, and ensure the feedback is delivered in helpful and private ways. These insights suggest ways to modify musical feedback systems, making them more effective tools in behavior change technology.

In the present disclosure, the terms “wellness” and “physical activity” are often used interchangeably; however, in practice these are two distinct concepts that are related. The term “wellness” has been used in the context of music models that were created based on participants' perceptions of musical wellness. However, when real-world behavioral data is involved in music or equations, the term “physical activity” is used to make it clear that the underlying studies, etc. focus on wellness in that context.

Wellness technology can, in certain situations, serve as an effective tool for encouraging healthier behaviors in users. These interventions can vary in complexity, with some systems deploy straightforward communication methods, such as sending text reminders, which are effective in addressing various health-related issues. Text reminders have helped participants with weight loss through constantly supplying information and promoting self-monitoring. These reminders have also been used to provide distractions, support, and information about smoking, helping more participants to quit after 6 weeks of intervention. Text systems have helped participants to better manage their diabetes, by promoting communication between patients and physicians. Information sent via texts as infrequent as once a week has also helped participants to better control their asthma. Some text-based reminders attempt to enhance engagement by incorporating elements like jokes and emojis. Text feedback has been further developed, creating agents to actively engage users in a dialogue about their wellbeing. Reflection Companion, for example, sent users prompts and follow-up questions encouraging users to reflect on their behavior. The study found this reflection encouraged users to remain engaged in the system, and them feel more motivated and empowered to be active. Other systems take a similarly straightforward approach of providing direct recommendations to encourage healthier behavior. MyBehavior provided users with personalized physical activity and dietary recommendations. While receiving these recommendations, the studies found participants spent significantly more time walking.

Indirect intervention and persuasion strategies have also been developed to encourage healthier behavior. Gamification seeks to enhance compliance by transforming behavior change into an engaging game. Through gamification, these approaches seek to make behavior change enjoyable and something users eagerly anticipate, thus increasing the likelihood of sustaining the desired behavior modifications. Gamified approaches have also explored ways to promote healthier behaviors across various domains, including physical activity, dietary habits, personal hygiene, hand washing, and managing chronic illness. One notable example of a gamified wellness application is Pokémon Go, estimated to have added 144 billion steps to the United States over a single month. Comparative work has investigated gamified approaches specifically for children, taking characters from animated children's movies and altering the avatar's actions based on the child's detected activity level. Other gamified approaches harness the power of competition among users, encouraging participants to vie with each other, allowing this competitive element to drive individuals toward healthier behaviors. While participants report enjoying competing with one another through simple comparisons such as leaderboards, competition is often more effective if participants compete with similar users indicating even simple competitions benefit from a level of underlying personalization.

On the other hand, ambient displays promote healthier behavior by incorporating health data into metaphors that reward users for making healthier choices. These ambient displays have creatively portrayed health information in various ways, such as transforming phone home screens into flourishing gardens, simulating aquatic ecosystems, and utilizing text bubbles. More recently, studies explored ambient displays that expand beyond one simple scene to tell a story. One such narrative-based ambient display is WhoIsZuki, which visually tells a story of a cute alien adventuring to find his brother. The story progresses as users meet their physical activity goals, encouraging physical activity so participants can learn what happens to the characters. Other narrative-based approaches integrate real-world stories. StoryMap allows families to post their own physical-activity-related stories, and read the stories posted by other users. While not perfectly an ambient display due to their textual nature, the results indicated these stories cause users to reflect on their own experiences, driving a sense of community that encourages users to be more active. In all, these ambient displays and narrative-based feedback systems show creative communication of wellness information allow the data to be more interesting, and possibly even foster emotional connections to the message.

Examples of phone-based ambient displays include the UbiFit Garden, where different flowers and butterflies demonstrated the participant's activity level and progress on their weekly activity goals. Bewell and Bewell+ followed the success of the UbiFit Garden. In the Bewell system, the scope was expanded beyond physical activity to also provide feedback on sleep and social interaction via an underwater ecosystem where various fish and their activities showed how healthy the user was in each of the three dimensions. BeWell+ provided several expansions focusing on more personalized score calculation and suggesting interventions to the dimension of wellness where users needed the most help. MONARCA system went beyond general wellness and specifically targeted individuals with bipolar disorder. Part of this system was an ambient display, which showed the factors affecting patients' conditions by changing the size and color of speech bubbles on the user's phone screen. Overall, these phone-based ambient displays may promote healthier behavior over a wide range of data types and through many metaphorical representations.

In addition to these digital representations, tangible ambient displays have been developed, including lights and color-changing clothing to encourage breaks for physical activity and shape-changing fabric to symbolize breathing patterns. On example is a health bar intended to help desk workers to take a break from sitting by changing the light color to red to encourage standing. MoodLight, a system that mapped the user's affect to the color of light, used EDA to determine the user's level of arousal, displaying warmer lights when the user is aroused, and cooler when the user is not. Another comparative example used an armband containing several colored rings, each correlating to a different range of heart rates. These were used to help users understand what phase of exercise they are in during a cardio workout. Each of these ambient displays provided data in an accessible manner, while becoming more pervasive than a simple phone screen. In a multi-week long study, 3D printed artifacts have even been used to communicate physical activity data. Users were presented with 5 printable objects ranging from a graph of their daily heart rate to a frog which changes size as the users are more active. The 3D printed artifacts served as useful conversation starters around the participant's activity, and even sparked a competition between participants to create the largest frog. Employing these metaphors allows technology to seamlessly blend into the user's environment, offering constant information that promotes healthier lifestyles.

However, these comparative examples primarily rely on users' visual perception. Other comparative examples have attempted to display information using other senses. However, limitations exist for most senses which prevent them from presenting complex information well. Haptics, for example, are used to passively portray information from many devices such as a computer mouse click, force applied to a joystick, or notifications on a phone or smartwatch. Researchers have embedded haptics into different locations such as floors. This use case could have unique applications, such as assisting the blind with self-navigation. Despite its prevalence, haptic displays are challenged by communicating highly complex data, often requiring support from other presentation modalities, such as audio or vision. Thus, haptics alone is not feasible for delivering data as complex as health assessments. Studies have been conducted to deliver smells using wearables. In an example, a necklace capable of producing basic scents such as a tea tree, peppermint, or a rose was used. The findings showed the perceived intensity of smell varied greatly among participants. A following study assigned different smells to indicate different notifications. Findings determined smells can trigger memories, stimulate, build expectations, identify and locate, and influence mood. However, olfaction has major limitations. First, it is very challenging to create specific smells. It is far harder to create smells than colors or music which can be assembled through their sub-components. Additionally, smell is a highly personal experience, and every person will interpret a smell differently, possibly triggering different moods and memories. As such, olfactory displays are not useful in displaying complex information. Of all the senses, communicating data through taste appears to be the least researched. It is possible to mechanically create tastes using electrical stimulation of the tongue. Through a 21-participant study, researchers proved smell, taste, and color can all be combined to alter the perceived taste of a drink. Data Cuisine created chocolate coffins with different fillings to represent causes of death. Despite the ability to invoke specific tastes, the sense is still rarely used to communicate data, possibly because triggering it requires eating food. This lack of feasibility may be due to sustainability, preservation, or the limited ability of humans to consume.

While vision is the most common mode for conveying health information, sounds and music have also demonstrated their effectiveness in communicating information and influencing behavior. Despite the use of ambient displays in invoking healthier behaviors, there is a noticeable gap in the comparative example on using automated audio systems to deliver analyzed health information and incite behavior change ambiently. This gap is particularly pronounced, as music's ability to spark behavior change in individuals is documented. In business settings, marketers frequently use changes in audio to invoke specific emotions, influencing customers into making specific shopping decisions. In a health setting, music therapy is a widely accepted form of psychiatric treatment, where clinicians use music and sounds to influence patients into better psychological well-being. Studies have even found music therapy more effective at treating depression than traditional psychotherapy treatments. As such, creating auditory wellness ambient displays may engage users differently than visual displays, as well as engage users that may be unable to receive information through visual cues, such as the visually impaired. In sum, although music is effective at invoking behavior change, it has been largely overlooked in systems that use health data in ambient methods to incite healthy behaviors.

The utilization of music and sonification in healthcare leverages music's capacity to stimulate various brain functions, including cognitive and emotional processing. This can be particularly beneficial for users, potentially offering advantages beyond traditional therapeutic approaches. However, nearly all comparative health data sonification techniques are geared towards providing real-time feedback on immediate stimuli, while the present disclosure constructs musical models of an individual's healthy behavior that can be played at a later time without relying on sonifying data in real time.

Research into sonification for wellness has been used to inspire users. Studies have investigated using musical scales, comprised of ascending and descending notes, to enhance confidence during physical therapy. They have also utilized repeated recordings of waves and birds for mindfulness during meditation, ambient noise for mindfulness during walking, and sounds like wind, gears, and beeps to foster a sense of agency during exercise. Music's ability to influence users' mental state is also evident in the effectiveness of music therapy, a field that employs music for patients' needs, through listening, performing, and composing. Music therapy has demonstrated effectiveness in the treatment and mitigation of several diseases. Reviews of music therapy research found listening to music can help reduce anxiety regardless of context, individuals with autism spectrum disorder use music to overcome social barriers by better communicating their thoughts and feelings, and depression as a supplement to traditional treatment, among other challenging health concerns. Given these applications and its success in complex treatments, it is notable that sonification's applications have remained relatively limited according to the comparative examples.

Indeed, sonification's applications in promoting wellbeing often revolve around representing real-time physiological changes. In the context of physical therapy, these changes frequently emanate from muscle or body movements during rehabilitation. These types of sonification can communicate data through different ways, including deviating from the expected conclusion of a song representing the squats a participant is doing or changing the music tempo while the participant is walking and lifting objects. Studies have also sonified physiological signals, such as EEG, pulse, and respiration. These studies use synthesized and symphonic music, which effectively engages users despite the inherent complexity of translating muscle movements and internal physiological cues into auditory experiences.

There exists a need, given music's proven ability to encourage both physical movement and improved internal physiological states, for sonification as a means to promote everyday physical wellbeing.

### Experimental Materials, Methods, and Results

To explore the feasibility of communicating wellness data through music, a 76-day within-subject study was conducted, during which participants received one month of musical feedback followed by one month of textual feedback on their activity levels or vice versa. Subsequently, participants' behavior was analyzed using data from Fitbit activity trackers and self-reported surveys to assess the effectiveness, shortcomings, and potential avenues for improvement in the practical application of musical feedback.

Participants were recruited through email and word of mouth. All participants attended a 1.5-hour onboarding meeting with the research team upon enrollment. During this session, they received detailed information about the study, completed the music modeling surveys, and were provided with a Fitbit Sense device. All Fitbits were linked to an account managed by the research lab, ensuring anonymous data access. Following the onboarding process, participants were requested to submit a week's worth of Fitbit data without receiving any feedback. This initial data collection week established a baseline for each participant, supplying the essential background data necessary for the operation of the activity level calculation. Participants were given a two-week window to provide this data, allowing for potential technical issues and the adaptation period required for syncing their Fitbit devices. Failure to comply within this timeframe resulted in removal from the study.

After completing the initial onboarding period, participants received two months of feedback on their activity levels. In a randomly chosen order, the feedback was provided to them through 31 consecutive days of surveys. These surveys presented either a musical or textual representation of their activity level. The approach for embedding activity level data into the music is explained in more detail below.

Textual feedback was chosen as the control condition due to its case of use and role as a foil to the experimental music condition. Feedback delivered by text is very clear, presenting one message with little to no interpretation. It is also widely used, with a literature review of mHealth behavior change techniques reporting that 47% of reviewed systems provide feedback through SMS or email. Other systems for promoting physical activity are slightly less interpretable, such as ambient displays conveying information through metaphors. While less direct than blatantly stating the wellness information, users can still verify their impression by visually analyzing the display. Musical feedback, however, is even less interpretable than ambient displays. The musical feedback was intended to deliver an emotional message about the users' level of physical activity. The musical feedback is then intended to encourage the user to reflect on why their physical activity made the music feel a certain way, ideally creating a sense of intrinsic motivation. Thus, text was used as a baseline because it is simple for users to understand, and its blunt and direct nature contrasts with the music.

The feedback surveys were delivered to participants daily at 6 p.m. local time through email and were hosted on the Qualtrics platform. Within each daily survey, participants were first asked to assess their valence and arousal using a 5-point Likert scale, which aimed to gauge their emotional state. Following this, they received information about their physical activity level. In the case of the music surveys, participants were prompted to identify which activity level they believed the accompanying song correlated with, while in the textual representation surveys, participants were simply informed of their activity level. Upon receiving and completing 62 days of these daily surveys, participants were invited to participate in a final survey. This concluding survey asked participants to reflect on their overall experience and share their thoughts, opinions, and insights gained throughout the study.

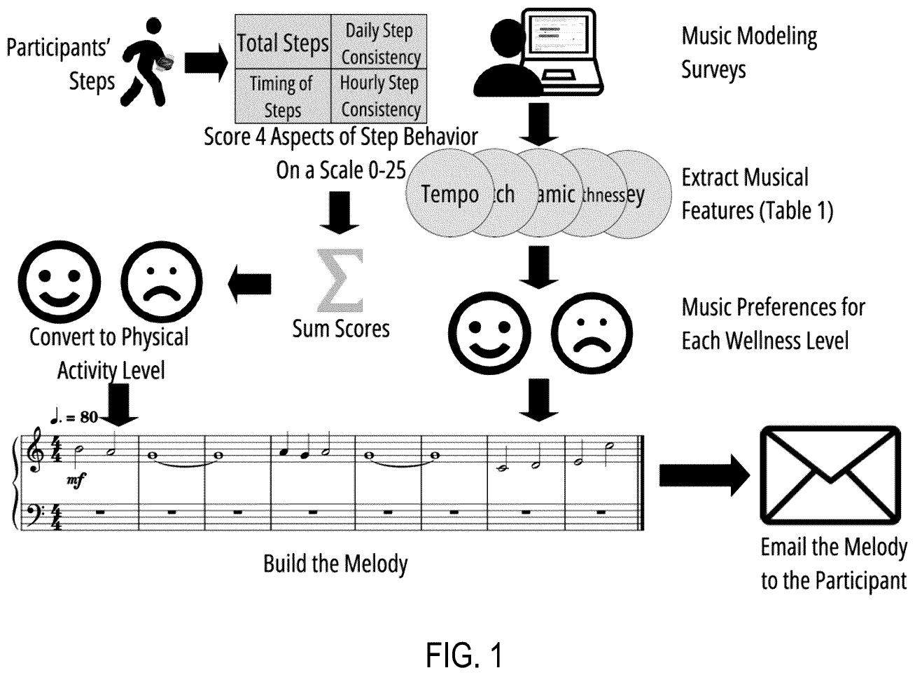

To explore music as a method to communicate wellness status, a procedure to convert a participant's data into music was developed. The approach consists of two primary steps. First, personalized music models were generated for individual users according to their perceived wellness of musical melodies at baseline, allowing the system to convey five distinct wellness levels. Then, Fitbit data from users was collected and an activity-evaluation algorithm was applied to convert their steps into one of these five physical activity levels. The resulting physical activity level is then translated into music and emailed to the participants. This approach is visually illustrated in FIG. 1 and further explained in the below. In particular, FIG. 1 shows an example approach to converting physiological data into sonified wellness information. The approach includes modeling participants' perceptions of wellness in music and converting physiological data into physical activities. One example for creating the musical models, in which participants listen to music with altered features and state how “well” they sound, will be described in more detail below. This is used to generate models for five distinct physical activity levels. Fitbit step data is then collected from participants and scored to one of five activity levels. The assigned physical activity level is then converted to music, and sent to the participant.

### Music Model Generation

These methodologies for creating the musical models can include a system that converts biobehavioral signals to musical models to convey the status of health seamlessly. One challenge presented is, however, that interpretation of music is highly subjective. A melody that sounds exhilarating to one may sound boring to another. The present disclosure, therefore, 1) identifies individual factors that affect one's impression of music healthiness, and 2) identifies what music characteristics relate to the perception of healthiness. A two-step approach is developed to achieve this goal. In the first step, a survey is created and applied to acquire users' general impression of music healthiness. In the second step, customized melodies are built for each user based on the combination of music preferences acquired in step one. These melodies were delivered to 55 participants over 31 days, who were then asked to rate the perceived healthiness of each melody. The patterns in the collected data were then analyzed to identify factors that affect users' characterization of healthiness in music.

In support of the musical model generation, a study was designed where participants were asked to complete the model creation process. During this process, users completed two surveys which were deployed over Qualtrics and delivered to the participants through their university email. The first contained audio files of melodies manipulated using the musical features: tempo, pitch, key, dynamics, and smoothness. After listening to each file, participants were asked to provide the wellness level (provided through a five-point Likert scale containing a range of unhealthy and healthy options) that best represented the tune. Those responses were then used to combine musical features and make healthy and unhealthy-sounding music for each individual participant. The generated melodies were deployed in personalized surveys in which participants provided their interpretation of the melodies.

This approach involved evaluating the participant responses and models on an individual level and combined with all other participants. The personal level allows one to determine how well the user could interpret music allowing us to create their personalized model, while the group analysis lets one determine the uniqueness (or lack thereof) of each model. In determining wellness, five distinct wellness levels were defined, each represented by a different point on the Likert scale. These five levels depict the varying statuses of healthiness ranging from very unhealthy (1) to very healthy (5). The five-point Likert scale was chosen based on a music ambient display that assessed musical features in affect using a similar scale.

These wellness levels are highly subjective, as they exist solely in the participant's interpretation of the music. Even when one attempts to incite specific health levels, the exact score is the user's decision. The decision to describe health in this manner was done intentionally, to ensure a system that is applicable to all types of wellness, rather than one that influences participant responses by asking them to consider a specific type of health in most questions. Using this method, once the user's models are complete, real health data could be applied to the user's interpreted wellness levels. As an example, a common daily goal is to walk 10,000 steps. These musical models could be deployed so that with every additional 2,000 steps, the melody depicting the healthiness of the individuals' steps changes to a melody that the user associates with a higher wellness level. As the user gradually walks more throughout the day, they will hear increasingly healthier-sounding music.

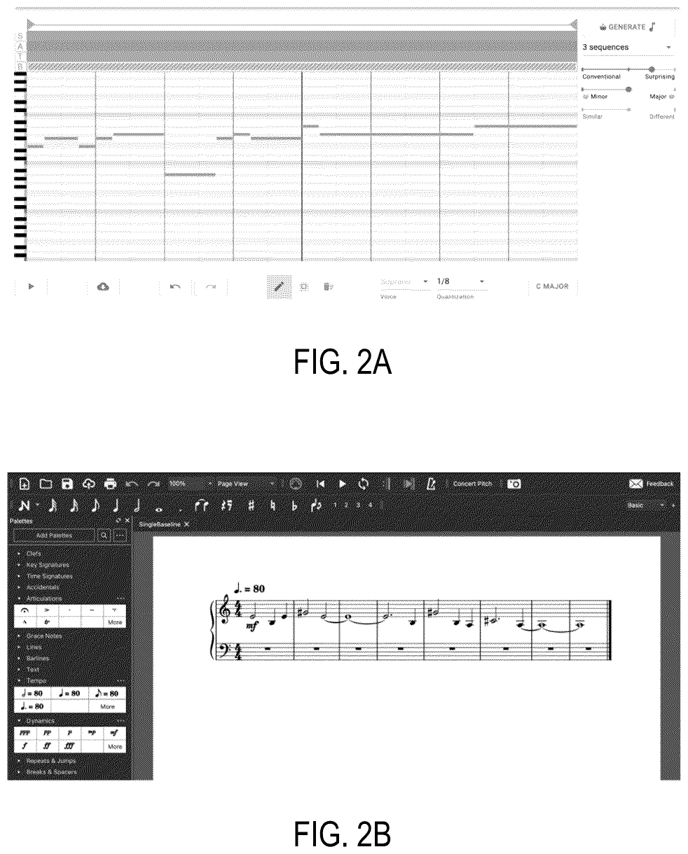

The method relies on manipulating musical characteristics to represent health data. Several musical elements were manipulated throughout the study. Table 1 contains the musical terms relevant to this paper and their definitions. All music utilized in this study was created using Bach CoCoCo, which generates melodies using DNNs while accepting inputs from users. Bach CoCoCo (FIG. 1a) was chosen to generate the music because it uses AI to create the music but still gives users control over the creation process. For this study, the most important feature allows for the creation of multiple melodic lines that consider the other lines to form a pleasing melody but can be created one at a time. This feature was essential to creating three-lined melodies because it meant that a line that fails to meet all the criteria could be recreated without needing to scrap the other existing melodic lines. FIG. 2A illustrates an example sample melody generated using Bach CoCoCo, and FIG. 2B illustrates the melody from one survey, created in MuseScore.

TABLE 1
Relevant Musical Terms and Their Definitions

| Music | |
|---|---|
| Term | Definition |
| Notes | The building blocks of music. notes |
| | represent tones and their duration. |
| Melody | A sequence of notes in a pleasing manner. |
| Tempo | The speed of music. |
| Dynamics | The volume of music. |
| Pitch | How high or low a note is. |
| Octave | A collection of sequential notes after which |
| | the name of the note begins to repeat. |
| Key | Specifies specific tones that should replace |
| | an adjacent tone throughout the melody. |
| Staccato | Indicates that notes are shortened, leaving |
| | a longer break between each pair. |
| Legato | Indicates that notes are lengthened, leaving |
| | little to no space between each pair. |

Once a tune was generated, it was evaluated simply to ensure the tune contained a melody. To contain a melody, it was required to meet three criteria. First, the melody had to have at least as many new notes as musical measures. This rule existed to prohibit tunes that only played a small selection of notes, or held a single note for a majority of the tune. Second, no notes could obviously clash, ensuring that the melody would not be inherently unpleasant to listen to. Lastly, the melody had to have multiple occurrences of notes that would change when the melody is transposed from the key of C-major to C-minor, ensuring that this musical feature included in the personalized models would be relevant for the music. If a melody did not meet these criteria, Bach CoCoCo generated a new melody.

If a melody met the criteria, it was recreated in MuseScore, as shown in FIG. 2B. MuseScore is a sheet music editor with a free-to-use version that provides access to a wide variety of music traits. This allowed for the easy conversion of melodies from Bach CoCoCo to a format granting greater access to modifications such as dynamics, tempo, and instrumental changes. It also provides an option to export sheet music to an MP3 so the music could be embedded into Qualtrics surveys without requiring any effort from the participant other than opening a survey.

A survey was created consisting of several audio clips, each changing a different factor (tempo/speed, dynamics/volume, major vs. minor keys, etc.). After each audio clip, participants were asked to rate the clip on a Likert scale depicting their impression of the healthiness the music clip would represent. Participants were also presented with clips of different musical instruments and were asked to rank them according to the type of health energy they felt those instruments represent.

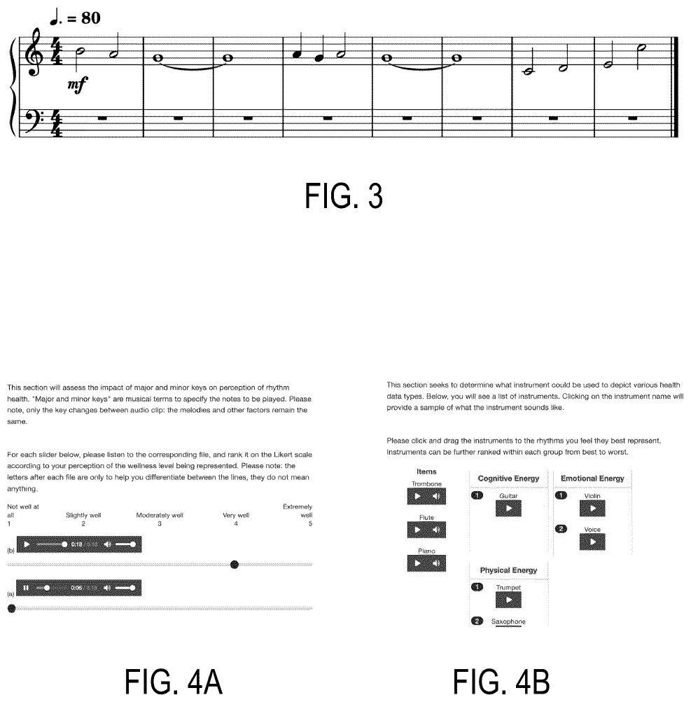

The goal was to identify the musical variables that created the impression of healthiness or unhealthiness in melodies. Split into several sections, the survey contained MP3 files of the same tune each altered by changing a single musical trait each time. For example, one audio file contained the melody in a major key and the following contained the same melody in a minor key. An unaltered version of the melody can be seen in FIG. 3. After each audio file, users were asked to consider that the music represented health data and to fill out a five-point Likert scale demonstrating the wellness level they believed the music represented. FIG. 4A illustrates an example interface for assigning health scores to different levels of smoothness.

To determine the musical features, a literature review of sonification in physical therapy was used, which identified the six most common auditory alteration categories to be: event-driven, loudness-related, timbral, pitch-related, spatial, and temporal. In the study, not all of these categories are possible. Event-driven changes trigger when a condition is met, and is not practical to deploy in the study because participants are receiving the data through a survey completed on their own time. Similarly, it was not possible to alter the spatial features because the sound will originate from the user's device. Lastly, timbral changes were not included because they may alter the quality of the audio, and lowering the quality could cause other features to become difficult to understand. Therefore, in the study five musical features: tempo, pitch, key, dynamics, and smoothness were manipulated in the survey. Table 2 briefly explains each feature.

TABLE 2
Musical Notation and Real World Equivalency for the Musical Features in the Study

| | | |
|---|---|---|
| Musical Notation | Real World Equivalency | Feature Name |

Tempo

| | | | |
|---|---|---|---|
| Eighth Note = 80 | 49 seconds | (40 BPM) | Very Slow |
| Dotted Eighth Note = 80 | 33 seconds | (60 BPM) | Slow |
| Quarter Note = 80 | 25 seconds | (80 BPM) | Moderately Slow |
| Dotted Quarter Note = 80 | 18 seconds | (120 BPM) | Moderately Fast |
| Half Note = 80 | 14 seconds | (160 BPM) | Fast |
| Dotted Half Note = 80 | 10 seconds | (240 BPM) | Very Fast |

Dynamics

| | | | |
|---|---|---|---|
| PP/Pianissimo | 63.4 | dB | Quiet |
| MF/Mezzo-Forte | 71.5 | dB | Middle |
| FF/Fortissimo | 85.5 | dB | Loud |

Key

| | | | |
|---|---|---|---|
| C Major | 329.63, 440.00, & 493.88 | Hz | Major |
| C Minor | 311.13, 415.30, & 466.16 | Hz | Minor |

Pitch

| | | | |
|---|---|---|---|
| C2 | 65.41-123.47 | Hz | Very Low |
| C3 | 130.81-146.94 | Hz | Low |
| C4 | 261.63-493.88 | Hz | High |
| C5 | 523.25-987.77 | Hz | Very High |

Smoothness

| | | | |
|---|---|---|---|
| Staccato | 1.11 | seconds | Staccato |
| Not Staccato | 2.22 | seconds | Legato |

Tempo: Six different tempos were included in the survey. The musical notation for tempo states how many of a certain type of note will be played in a minute. Table 2 shows all six tempos, both in musical notation and file length in seconds. This feature was included due to the temporal changes found in the literature review.

Pitch: The files were manipulated so that the majority of a melody falls within a specific octave (range of pitches). In this survey, the notes ranged from C2 (low) to C5 (high). This range was selected as each range sounds unique but is not painful to listen to. Table 2 shows the octaves used in this study, and their respective frequency ranges. This feature was included as it lies within the pitch-related category found in the literature.

Key: The study used melodies in the keys C-major, and C-minor. Three notes differ between these two keys by a half step (a small change). Traditionally, minor keys are thought to make music sound sadder, however, this is not always the case. A famous example of this is Survivor's “Eye of the Tiger” is in the key of C-minor, the same key as this study. Table 2 shows the frequency of the impacted notes in octave C4. Key was included as another interpretation of altering the pitch-related category. The literature review defined this category as an “increase or decrease in perceived audio frequency.” While this variable may mean pitch, it could also indicate the key of the music, which changes the frequency of three notes rather than all seven.

Dynamics: Three different dynamics, namely pianissimo (quiet), mezzo forte (medium), and fortissimo (loud) were used. These dynamics were selected because they were extreme enough values to sound different, but not so extreme that the music became challenging or painful to hear. Sample decibels recordings for each dynamic are shown in Table 2. These measurements were made from the same computer, with the speakers set to the same percentage. Exact decibels will vary between recordings based on these settings. Dynamics was included in the study as an example of the loudness-related variables detected in the literature.

Smoothness: Users listened to the melody where every note was staccato, and one where the notes were not. The opposite of staccato is legato (smooth), not normal notes that were used in this study. However, during development, it was noted that the exported MP3 files of legato melodies had no apparent noticeable difference compared to melodies where the notes were both not staccato and not legato. Therefore, the personalized models use staccato and not staccato rather than staccato and legato, although the term “legato” will be used for simplicity. The length of the same sustained note, both staccato and not are shown in Table 2. Smoothness was included in the study as another example of temporal changes. While the speed of the song overall was determined as a strong example of a timing change, the amount of space between notes might also have a significant impact on a user's perception of health.

Additionally, the study also seeks to determine if evaluating multiple lines at once impacts the user's ability to depict health levels. The motivation for doing so was that music can, theoretically, express multiple health levels simultaneously as melody with multiple lines. To differentiate the different lines, each was designed to be played by a different musical instrument. In this initial survey, users were asked to listen to the same audio clip but played by different instruments. The possible instruments are flute, guitar, piano, saxophone, trombone, trumpet, violin, and voice. Participants were asked to classify each instrument as best representing a specific type of health energy: cognitive, physical, or emotional as seen in FIG. 4B. In addition to sorting between the classified energy groups, users could sort within each group, specifying which instruments better classify each type of energy than other instruments within the same group. While the instrument question was always asked last, the sections focusing on musical features were randomized for each participant, and the audio files within each section were randomized as well. This was done to minimize the influence between audio files.

Participants' ratings of healthiness impression were used to build customized models, and participants were further evaluated in their ability to distinguish the healthy music combinations from unhealthy ones. Multiple features were combined based on the user's responses to the first survey. Herein, personal models are defined to be the collection of what each participant chose as the healthiest and unhealthiest sounding feature for each musical characteristic. Building these models on a personal level was inspired by inScent, which let participants decide what meaning each scent should have. Much like smell, music is a highly personal experience with every person enjoying different types of music. As such, it was decided that a musical-based system should be personalized so that it can be accurately and easily understood.

For each musical feature in survey one, the option participants chose as the healthiest and the unhealthiest were identified. For example, if “very fast” was ranked as the healthiest of all tempos, and “very slow” as the least healthy of all of the tempos, those tempos were recorded as the settings in the healthy and unhealthy models. In the case a user provided the same level of healthiness for multiple choices, the system would decide the respective feature from among these. To prevent overlap between the healthy and unhealthy models, the features were not chosen randomly. Instead, it began by looking at one end of the spectrum, and worked to the other end, taking the first value it reached that was included in the tie. For each feature, determining healthy and unhealthy began on opposite sides of the spectrum, so overlaps are not possible.

To demonstrate, imagine a user ranked the pitches such that “very low” and “very high” were tied for the unhealthiest option, while “low” and “high” were tied for the healthiest. The method began by finding the feature for the healthy model, starting at “very high.” Because “very high” was not a viable option, it will move to “high” (which is a possible choice), and save that as the healthy pitch. It would then repeat this process for the unhealthy model, starting with “very low.” Since the “very low” pitch was rated as one of the unhealthiest features, it would be selected. This would result in “high” being the pitch for the user's healthy model, and “very low” being the pitch for the user's unhealthy model.

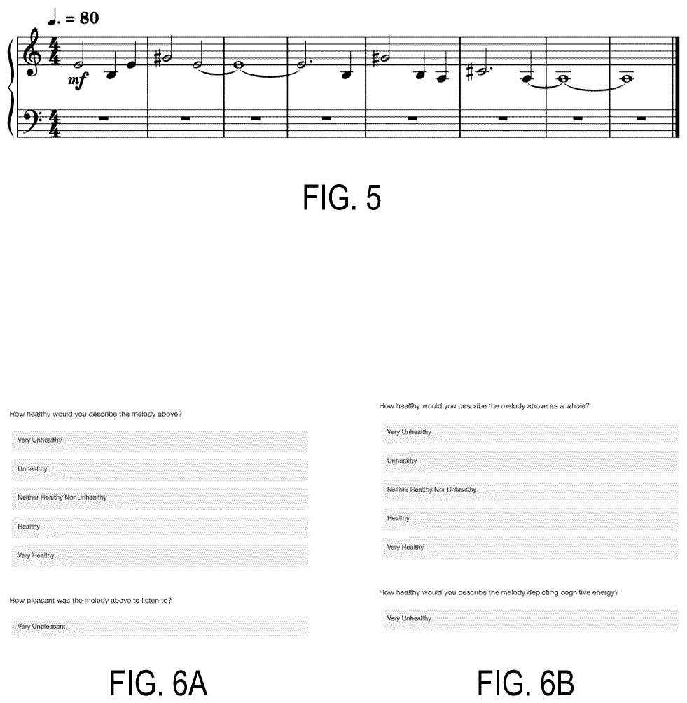

Once the personalized models were created, the interpretation survey was generated using them. The goal of this survey was to determine how the user will interpret music where the features are combined. The audio files in this survey use elements from the user's healthy and unhealthy models on the same melody. An unaltered melody from the second survey is shown in FIG. 5. Every audio clip contains either the healthy or unhealthy setting for each musical feature described in the preferences survey. Participants analyzed these audio files using a five-point Likert scale, the same method as the preferences survey. Additionally, for this study, an additional question was added to this survey. After each audio file, participants were asked to complete a 5-point Likert scale for how pleasant the melody was. These questions were included because it was suspected that pleasantness would serve as a strong indicator of perceived healthiness. This portion of the survey can be seen in FIG. 6A.

The interpretation survey was comprised of two melodies: one with a single melodic line, and another with three. The single-line melody was always played on the piano. It assessed how users perceive wellness when all musical factors from the first survey were combined. In some instances, all factors indicated the same level of wellness, while other clips assessed how participants will perceive conflicting information. In all, there were seven audio clips containing a single melody line. One contained all “healthy” features, one contained all “unhealthy” features, and five contained conflicting information. For ease of reference, these will henceforth be called composition H, U, A, B, C, D, and E respectively. The settings that made up each composition can be seen in Table 3. When discussing healthy and unhealthy features these are the respective options for each musical element that were saved in the user's model to indicate healthy or unhealthy. Using the previous example, “very slow” would be the model's unhealthy tempo feature, while “fast” would be the model's healthy tempo feature.

TABLE 3
Features of All Seven Compositions

| Music Feature | H | U | A | B | C | D | E |
|---|---|---|---|---|---|---|---|
| Tempo | Healthy | Unhealthy | Unhealthy | Healthy | Healthy | Unhealthy | Unhealthy |
| Pitch | Healthy | Unhealthy | Unhealthy | Healthy | Unhealthy | Unhealthy | Healthy |
| Key | Major: | Major: | Major: | Major: | Major: | Major: | Major: |
| | Healthy | Unhealthy | Healthy | Healthy | Unhealthy | Unhealthy | Healthy |
| | Minor: | Minor: | Minor: | Minor: | Minor: | Minor: | Minor: |
| | Unhealthy | Healthy | Unhealthy | Unhealthy | Healthy | Healthy | Unhealthy |
| Dynamics | Healthy | Unhealthy | Healthy | Unhealthy | Healthy | Unhealthy | Healthy |
| Smoothness | Healthy | Unhealthy | Unhealthy | Unhealthy | Healthy | Healthy | Unhealthy |

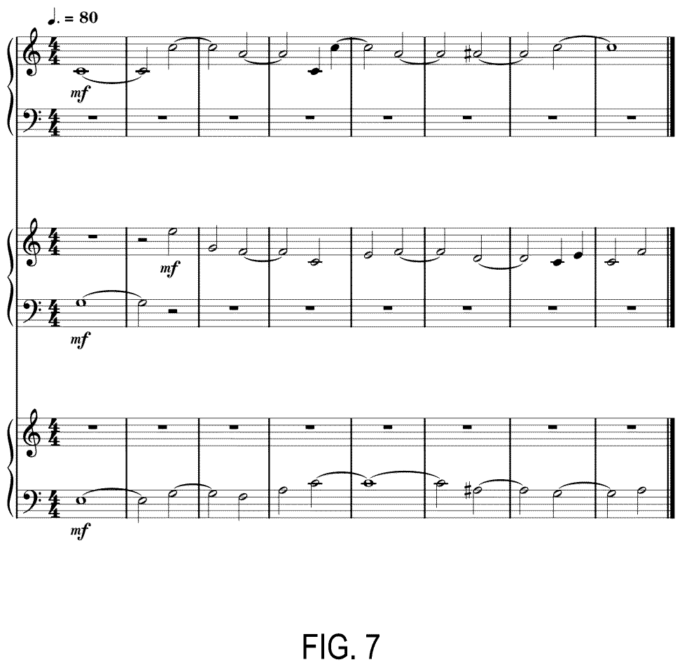

The second melody, shown in FIG. 7, was a three-line melody, where three different sets of notes were played simultaneously. During the study, users were asked to listen to this melody four different times, twice where each line was using all the features from the healthy model, and twice again with unhealthy features. For each level of healthiness, it was played once solely on the piano, and again by a different instrument that the user specified in the previous survey. When each line was represented by a different instrument, users were asked to further break down the melody, and state the wellness level of each type of energy. The three-lined melodies were played on both the piano and instruments so any inconsistencies compared to the single-line melodies can be determined to be a result of the instrument changes, or the change to multiple lines. As with the other question types, a picture of the survey asking questions from this section is shown in FIG. 6B. While the single-line melodies were always played before the three-line melodies, all compositions within each group were provided in a random order for each participant.

For this study, 55 participants at a large university in the mid-Atlantic United States were recruited, and 52 (13 male, 39 female) were included in the data analysis. In the first round, participants were sent the general healthiness impression survey to fill out. Responses to the first survey were then used to generate models and melodies based on each participant's preferences. These melodies were incorporated in an individualized interpretation survey and sent to participants for rating. The surveys were deployed and left open for participants to complete at their own pace over 31 days. During this time, 55 participants completed the general healthiness survey (henceforth called survey 1) and 50 completed the individualized interpretation survey (henceforth called survey 2). Two responses from each survey had to be removed because participants provided the same answer for every question, indicating they may have been focused on completing the study for compensation rather than providing useful data. The participants with insufficient answers in the first survey had to have their second survey removed as well because the first survey's responses impacted the creation of the second. However, the survey 1 responses for the problematic survey 2 participants were left in for data analysis because even if the participant did not take the second survey seriously, they appeared to do so with the first. Additionally, one response to each survey had to be removed, because a participant took the study twice. Therefore, during data analysis, 52 responses to the first survey and 45 for the second were included. Upon completing survey 2, the participants were automatically flagged to receive $10 in compensation.

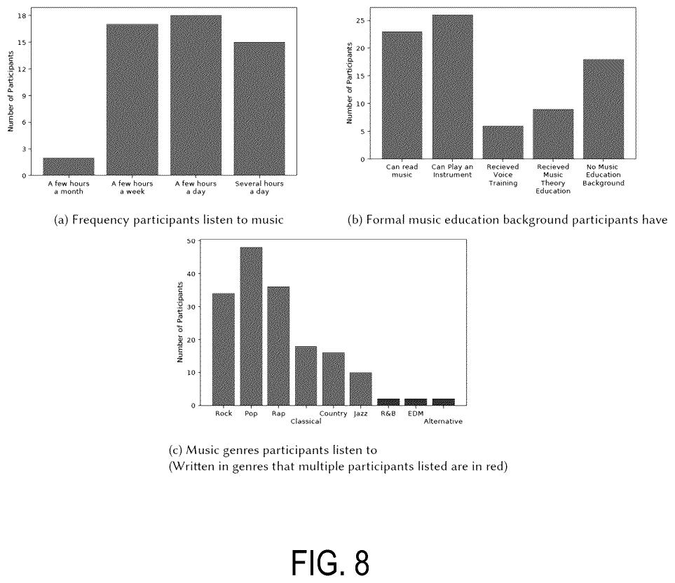

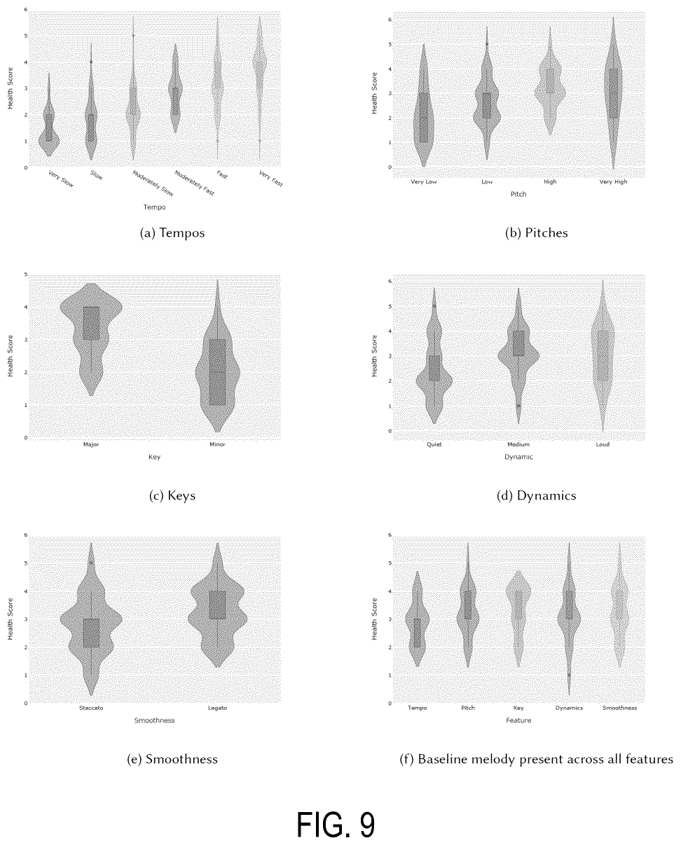

To understand the effect of demographics on music preference and interpretation, information about race and ethnicity was also collected. Thirty-two participants self-identified as white, eighteen as Asian, five as Black or African American, and one as Hispanic; thirty-four participants identified as not Hispanic or Latino, twelve as non-Black, non-White Hispanic or Latino, eight as White Hispanic or Latino, one each Middle Eastern and Asian, and lastly two participants opted not to provide their race. Participants were further asked about their music background because theories identify music background as a potential influencing factor of music taste. In particular, users provided musical information regarding music genre preferences, music education, the frequency with which they listen to music, and activities they do while listening to music. FIG. 8 shows the musical demographics of participants. The majority of participants listened to music every day, and more than half indicated they can read music and/or play an instrument. The most popular genres of music were pop, rap, and rock.

To analyze the effectiveness of and future considerations for sonification-based health applications, the following questions were investigated. R1: What music features (single or combined) make the melodies to be perceived as healthy or unhealthy? R2: How well can the combination of multiple aspects of health (e.g., physical, emotional, and cognitive) be expressed in one melody? Is the healthiness of a single health aspect more easily communicated with the users than multiple? R3: How common are the healthiness impressions of music features among participants? R4: What external factors affect the perceived healthiness of music? To what degree do demographics, music background, and frequency of listening to music affect participants' impression of music healthiness?

Regarding question R1, to create personalized melodies aligned with users' music healthiness impression, one must first identify how the healthiness of each musical feature was perceived. To begin, participant responses to the general healthiness impression survey were analyzed. In the first survey, participants provided their impressions of how each musical feature impacted the healthiness sound of the melody. To provide these impressions, participants ranked the features on a five-point Likert scale representing very unhealthy (1) to very healthy (5). In the following, participants' responses to each feature were analyzed and compared to the other features within the same category. In doing so, the focus was on finding significant differences between responses to the various variables and defining the trends within each musical feature. The mean scores and distribution of responses for each feature are shown in Table 4 and FIG. 9 respectively.

TABLE 4
Summary of Health Scores for Each Individual Musical Feature

| | | |
|---|---|---|
| Feature | Mean | Standard Deviation |

Tempo

| | | |
|---|---|---|
| Very Slow | 1.442 | 0.608 |
| Slow | 1.788 | 0.776 |
| Moderately Slow | 0.847 | 0.715 |
| Moderately Fast | 2.865 | 0.715 |
| Fast | 3.173 | 0.923 |
| Very Fast | 3.712 | 0.923 |

Dynamics

| | | |
|---|---|---|
| Quiet | 2.442 | 1.110 |
| Middle | 3.096 | 0.852 |
| Loud | 3.039 | 1.084 |

Key

| | | |
|---|---|---|
| Major | 3.327 | 0.785 |
| Minor | 2.019 | 0.852 |

Pitch

| | | |
|---|---|---|
| Very Low | 2.096 | 1.034 |
| Low | 2.635 | 0.908 |
| High | 3.231 | 0.783 |
| Very High | 3.039 | 1.137 |

Smoothness

| | | |
|---|---|---|
| Staccato | 2.769 | 0.983 |
| Legato | 3.25 | 0.86 |

The tempo ratings followed the trend that faster is healthier, as shown in graph (a) of FIG. 9. A Kruskal-Wallis test comparing responses for each feature within tempo provided significance results (H=156.230, p=6.293×10⁻³²), indicating there were differences in how participants interpreted the various melody speeds. The very slow tempo provided the lowest average score of any feature in the study. Health score gradually increased as the tempo gets faster, with the fastest tempo being the one participants perceived to be the healthiest. As the mean score increased, the standard deviation tended to as well. This may indicate that while most participants considered faster tempos to be healthier, they disagreed on the degree of healthiness.

As with tempo, a Kruskal-Wallis test showed there are differences in the interpretation of healthiness based on pitch (H=34.419, p=1.616×10⁻⁷). However, the pitch did not follow as clear of a trend as the tempo, as shown in graph (b) of FIG. 9. Generally, participants perceived higher pitches as healthier. However, when the pitch becomes too high, the impression of healthiness drops. As a result, the “high” pitch variable was perceived to be the healthiest value. This is notable because the value referred to as “high” is significant in music as well. This octave, beginning with a note called “Middle C” is considered to be the center of the music scale, and it commonly appears in music. Therefore, while participants perceived higher pitches as healthier, they may also have interpreted common pitches as healthier.

Key also had a significant impact on participants' interpretation of healthiness. A Kruskal-Wallis test between the major and minor keys indicated a level of significance (H=40.424, p=2.044×10⁻¹⁰). Participants strongly associated the major key with a higher level of healthiness, as shown in graph (c) of FIG. 9. On average, the switch from major to minor decreased the perceived healthiness by 1.3 points, a change larger than all other features except for tempo.

Similar to the previously discussed musical features, changes in dynamics also resulted in changed impressions of health (H=12.256, p=0.002). As shown in graph (d) of FIG. 9, participants considered quiet melodies to be less healthy than those at a loud or middle volumes (H=12.188, p=4.809×10⁻⁴). This is particularly notable because a Kruskal-Wallis test showed no significant difference between the middle and loud dynamics (H=0.031, p=0.860). Thus, while participants perceived quiet melodies as unhealthy, they did not perceive significant differences between the healthiness of middle and loud dynamics.

Lastly, smoothness also impacted users' perceptions of music's healthiness (H=6.304, p=0.012). As shown in Table 4 and graph (e) of FIG. 9, participants generally found legato models healthier than staccato models. That is to say, smoother melodies appeared to be perceived as healthier.

The analysis of the first survey was concluded with a comparison across each feature category. Within each set of features, there was one musical composition that was present in all five categories. This composition was included to ensure participants responded similarly to a melody if they encounter it multiple times. Between the five feature categories, the baseline compositions were: moderately fast, high, major, middle, and legato. These compositions can be seen in direct comparison in graph (f) of FIG. 9. When comparing these musical features with a Kruskal-Wallis test, there was a significant difference (H=78.445, p=3.719×10⁻¹⁶), indicating user responses may not be consistent over time. However, when “moderately fast” was removed from the set, the significance disappears (H=2.313, p=0.510). Because there was no significance between these four baselines, one can assume participants perceived them the same way, indicating there is little to no difference between them. Due to the randomized order in which the baselines were presented, it is assumed that the tempo baseline was only impacted by the other tempo melodies being perceived as healthier. Therefore, that participant perceptions were determined to frequently remain stable, but may also vary given the context in which the melody is delivered.

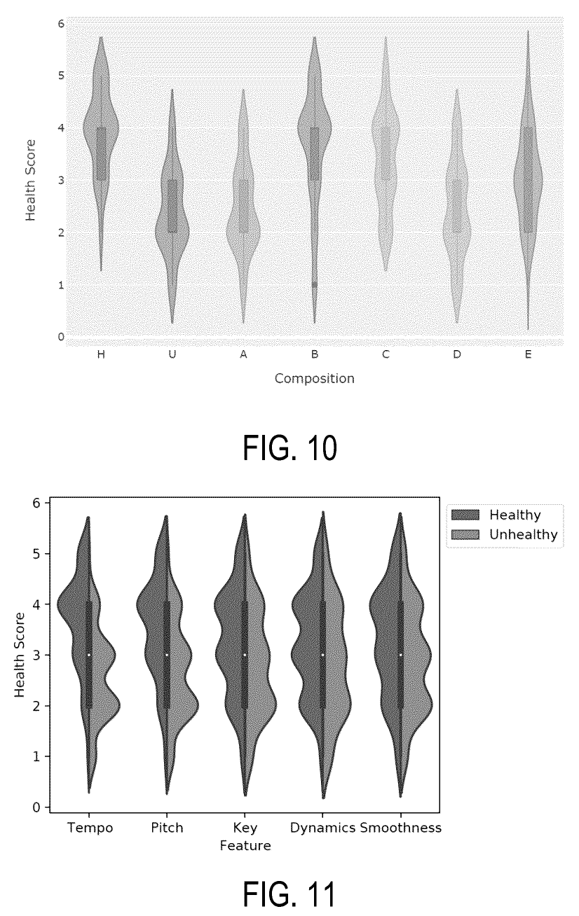

The analysis of the initial survey provided insights into how musical features impacted the perceived healthiness of music. To determine whether these effects remain when musical features are combined, the responses to the second survey were analyzed through the context of each of the 7 musical compositions described in Table 3. These compositions each combined features from users' healthy and unhealthy models described previously. These compositions were analyzed using a generalized linear mixed model. This allowed for a determination of the average impact each musical feature had on the overall healthiness and whether this difference was significant while reducing the impact of individual differences between the compositions. As such, the model used the participant's rating as the dependent variable, a binary representation for each of tempo, dynamic, pitch, smoothness, and key as either “healthy” or “unhealthy” as independent variables, and the composition itself as the random effect to minimize the impact of the different melodies had on one another. All of the mixed models were created using the statsmodels package in python. The exact scores of these compositions as a whole in analysis can be seen in FIG. 10 and Table 5.

TABLE 5
Summary of Participant Perceptions for
the Musical Compositions in Survey 2

| Composition | Mean | Standard Deviation |
|---|---|---|
| H | 3.911 | 0.793 |
| U | 2.378 | 0.806 |
| A | 2.467 | 0.842 |
| B | 3.622 | 1.093 |
| C | 3.378 | 0.912 |
| D | 2.422 | 0.917 |
| E | 3.022 | 0.866 |

It is to be noted that there were inconsistencies with the “key” variable. Due to an error in model creation, all participants were classified as considering the major key to being healthier than the minor key. In reality, 42 participants believed this, 5 saw no difference, and 5 believed the minor key sounded healthier. Due to the binary nature of this variable it was still possible to analyze the models by using the anonymous identifiers within the surveys to track which users received the wrong key and adjusting the analysis as necessary.

Similar to what results alluded to in the previous section, the analysis indicated tempo had the largest impact on the perceived healthiness of a combined melody. On average, healthy tempos were classified 0.778 points higher than unhealthy tempos, providing a significant difference (z=5.825, p=2.9×10⁻⁹). Pitch also provided a significant impact between healthy and unhealthy compositions (z=3.491, p=2.407×10⁻⁴). On average, a composition using a healthy pitch was classified 0.452 points higher than one using an unhealthy pitch. The last variable that significantly impacted the composition's perceived healthiness was key (z=2.111, p=0.035). Using the healthy key resulted in an average improvement of 0.194 points over the unhealthy key.

While the effect produced by smoothness was not significant (2=1.157, p=0.247), healthy smoothness was, on average, classified 0.146 points higher than unhealthy. Due to the lack of significance, participants likely did not associate changes in smoothness to hold much impact on healthiness. Dynamics also did not provide a significant impact (2=1.015, p=0.310), but on average healthy dynamics were classified 0.109 points higher than unhealthy dynamics. While this could mean dynamics do not have an impact on perceptions of health, it is believed that another explanation is more likely. In the study, the participants completed surveys on their own computers. It is possible that between the beginning of the first survey and the end of the second, participants changed the volume on their machines. This could have been done during a study or during the period between them. Regardless, it may have impacted how users perceived the various dynamics.

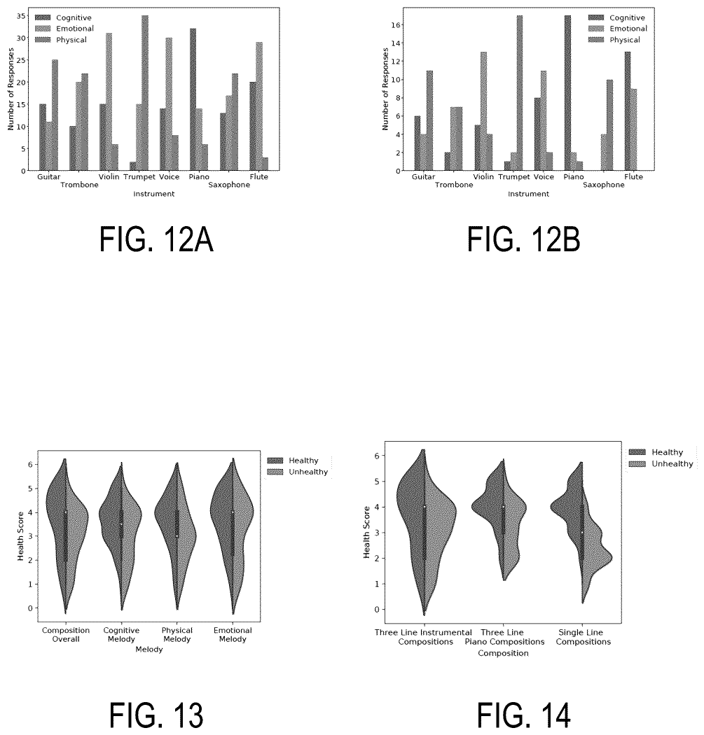

While definitive trends did exist within the data, exact interpretations varied greatly between participants, as will be discussed below. Thus, while one can create assumptions to predict how users will respond to different musical features, building personalized models remain necessary to portray the necessary information. A summary of results from this section can be seen in Table 6, and the distribution of compositions using the healthy and unhealthy options for each feature category are presented in FIG. 11. In Table 6, significance indicates that changing the musical element from healthy to unhealthy results in a lower perceived healthiness.

TABLE 6
Summary of the Impact Each Musical Element
Had on a Melody When They Are Combined

| Musical Feature | P-Value | Significant |
|---|---|---|
| Tempo | 2.9 × 10⁻⁹ | Yes |
| Pitch | 2.407 × 10⁴ | Yes |
| Key | 0.035 | Yes |
| Smoothness | 0.247 | No |
| Dynamics | 0.310 | No |

In examples, the present disclosure presents systems and methods of presenting health information related to multiple health factors such as physical, emotional, and cognitive in one single melody. This concept was implemented by developing three-line melodies, where each line is played by a different instrument. Each instrument represented a unique aspect of health (e.g., physical health), as specified by the participant in the first survey. These health aspects, represented as energy types in the survey, function the same way as the single-line melodies; they were manipulated through the user's personalized models to demonstrate various health levels. To test the feasibility of combining multiple health factors in one melody, user perceptions of instruments and the types of health energy they represented were first analyzed. The overall health (all three aspects) may be received through the three-line melodies.

In the first survey, participants were asked to listen to a melody played by various instruments. These instruments were categorized to represent one of three types of energy: cognitive, emotional, and physical. In addition to categorizing the instruments, users could sort them, indicating the instruments they felt “best” represented the energy compared to all other instruments. FIG. 12B shows the distribution of how many times each instrument was assigned as the best instrument in each category. Cognitive and physical energy both showed a strong consensus with a clear preference for piano and trumpet respectively. While not as common as piano and trumpet, guitar and flute were frequently sorted into these categories as well. Emotional energy also showed instruments that are popular, although the difference is not as profound as trumpet and piano. In this case, participants often associated the violin and voice with emotional energy.

Although the association of instruments to emotional energy looked similar when ignoring rank (see FIG. 12A), there is one difference. Considerably more participants assigned the flute to emotional energy than cognitive energy, despite the fact that more participants ranked it as the best instrument for cognitive energy than emotional. This difference is notable because all instruments other than flute remained most commonly associated with the same energy type. However, flute is the third most popular choice for emotional energy in both representations. This substantial drop, may not be due to the flute itself, but due to the apparent uncertainty that exists within emotional energy, especially when compared to cognitive and physical energy which have clearly defined peaks.

Overall, these results showed trends in associating instruments with types of energy. While most energy types were associated with multiple instruments, cognitive and physical both had well-defined instruments that were most commonly associated with them. Yet, emotional energy was far less definitive.

After establishing their instrument preferences, participants listened to and interpreted the three-line melodies. The study included four of these melodies, two using instruments and two using only the piano. Participants were only asked to interpret individual levels when using the instrumental compositions. However, the piano compositions were included to act as a baseline, so any differences between the instrumental composition and single line could be narrowed down to being a result of the instruments, or the transition from one line to three.

The healthiness level was intended to be identical across the overall melody and each individual line. Generalized linear mixed models were created to test for a difference between all three lines in both melodies. For these models, the participant's rating was again used as the dependent variable. The energy being depicted (cognitive, physical, emotional, and overall) was used as the independent variable. The participants themselves were used as the random effect because, in this specific analysis, there was a desire to ensure consistency within each user rather than isolating the impact of musical elements from each other. No significant difference was found between any pair of energy within the healthy or unhealthy composition. The distributions for all types of energy and perceived healthiness of the three-line instrumental melody and each individual line can be seen in FIG. 13.

The success of the three-line instrumental melodies lessens when comparing across different melodies. Using generalized linear mixed models, the instrument and piano three-lined melodies were compared to each other, and to the single-line melodies H and U which used the same musical features. Same as in the previous models, the user rating was used as the dependent variable. Meanwhile, the composition type (instrumental, three-line piano, and single-line) was used as the independent variable. The participants were again used as the random variable for these models. There was no significant difference between the two types of three-lined melodies (z=1.743, p=0.081), or the instrument and single-lined compositions (z=1.072, p=0.284). However, there was a difference between piano and single-lined compositions (z=2.815, p=0.005). The distributions of responses to each of these compositions can be seen in FIG. 14. These results indicate that the addition of instruments did not appear to impact the perceived healthiness, but the conversion from one line to three might have.

Thus, while instrument meanings may be generalizable, the three-line melodies may lead to different health perceptions than intended. Even though participants can accurately interpret healthiness from sonified data in single and three-lined melodies, further research is needed to determine whether users can accurately depict the same sonified data from single and three-line melodies that both use the same personalized models. Different unique personalized models may be required for the three-line melodies as well as the single-lined melodies. A summary of results discussed in this section can be found in Table 7. Significance indicates the two elements (different melodic lines in the top section, and different instrumental compositions in the bottom three rows) were perceived differently by participants.

TABLE 7
Summary of All Significance Tests Conducted
on the Three Lined Melodies

| Comparison | P-Value | Significant |
|---|---|---|
| Overall Energy & Cognitive Energy | 0.842 | No |
| Overall Energy & Emotional Energy | 0.464 | No |
| Overall Energy & Physical Energy | 0.842 | No |
| Cognitive Energy & Emotional Energy | 0.351 | No |
| Cognitive Energy & Physical Energy | 1.000 | No |
| Emotional Energy & Physical Energy | 0.351 | No |
| Three-Lined Instrument & Three-Lined Piano | 0.081 | No |
| Three-Lined Instrument & Single-Lined | 0.284 | No |
| Three-Lined Piano & Single-Lined | 0.005 | Yes |

To determine the commonalities among participants in their healthiness impression of music features, the models that were generated from the initial survey responses were analyzed, focusing on more frequent models that were common among participants as listed in Table 8. All models generated throughout the study are included in Table 9.

TABLE 8
Number of Occurrences and Settings for All Personalized
Models That Were Generated by Multiple Participants

| | | | | | |
|---|---|---|---|---|---|
| No. | | | | | |
| Partic- | | | | | Smooth- |
| ipants | Tempo | Pitch | Key | Dynamics | ness |

Healthy

| | | | | | |
|---|---|---|---|---|---|
| 3 | Moderately Fast | High | Major | Middle | Legato |
| 3 | Fast | Very High | Major | Loud | Legato |
| 2 | Very Fast | Very Low | Major | Loud | Legato |
| 2 | Very Fast | Very High | Major | Middle | Legato |
| 2 | Very Fast | Very High | Major | Loud | Staccato |
| 2 | Very Fast | Very High | Major | Loud | Legato |
| 2 | Very Fast | Low | Major | Quiet | Legato |
| 2 | Very Fast | High | Major | Middle | Staccato |
| 2 | Very Fast | High | Major | Loud | Legato |
| 2 | Moderately Slow | High | Major | Middle | Legato |
| 2 | Fast | Very Low | Major | Middle | Legato |

Unhealthy

| | | | | | |
|---|---|---|---|---|---|
| 4 | Moderately Slow | Very Low | Minor | Quiet | Staccato |
| 3 | Very Slow | Very Low | Minor | Quiet | Staccato |
| 3 | Slow | Very Low | Minor | Loud | Staccato |
| 3 | Slow | Very High | Minor | Loud | Staccato |
| 2 | Very Slow | Very High | Minor | Quiet | Staccato |
| 2 | Very Slow | Low | Minor | Loud | Staccato |
| 2 | Slow | Very Low | Minor | Quiet | Legato |
| 2 | Slow | Low | Minor | Quiet | Staccato |

TABLE 9
All Personalized Models Created During This Study

| | | | | | |
|---|---|---|---|---|---|
| No. | | | | | |
| Partic- | | | | | Smooth- |
| ipants | Tempo | Pitch | Key | Dynamics | ness |

Healthy

| | | | | | |
|---|---|---|---|---|---|
| 3 | Moderately Fast | High | Major | Middle | Legato |
| 3 | Fast | Very High | Major | Loud | Legato |
| 2 | Very Fast | Very Low | Major | Loud | Legato |
| 2 | Very Fast | Very High | Major | Middle | Legato |
| 2 | Very Fast | Very High | Major | Loud | Staccato |
| 2 | Very Fast | Very High | Major | Loud | Legato |
| 2 | Very Fast | Low | Major | Quiet | Legato |
| 2 | Very Fast | High | Major | Middle | Staccato |
| 2 | Very Fast | High | Major | Loud | Legato |
| 2 | Moderately Slow | High | Major | Middle | Legato |
| 2 | Fast | Very Low | Major | Middle | Legato |
| 1 | Very Slow | High | Minor | Quiet | Legato |
| 1 | Very Fast | Very Low | Minor | Middle | Legato |
| 1 | Very Fast | Very Low | Minor | Loud | Legato |
| 1 | Very Fast | Very Low | Major | Middle | Staccato |
| 1 | Very Fast | Very Low | Major | Loud | Staccato |
| 1 | Very Fast | Very High | Major | Quiet | Staccato |
| 1 | Very Fast | Very High | Major | Quiet | Legato |
| 1 | Very Fast | Low | Minor | Middle | Staccato |
| 1 | Very Fast | Low | Major | Loud | Legato |
| 1 | Very Fast | High | Minor | Middle | Legato |
| 1 | Very Fast | High | Minor | Loud | Staccato |
| 1 | Very Fast | High | Major | Quiet | Legato |
| 1 | Slow | Low | Major | Quiet | Legato |
| 1 | Moderately Slow | High | Minor | Middle | Legato |
| 1 | Moderately Fast | Very High | Major | Middle | Legato |
| 1 | Moderately Fast | Low | Minor | Loud | Legato |
| 1 | Moderately Fast | Low | Major | Quiet | Legato |
| 1 | Moderately Fast | High | Minor | Middle | Staccato |
| 1 | Moderately Fast | High | Minor | Middle | Legato |
| 1 | Moderately Fast | High | Major | Middle | Staccato |
| 1 | Fast | Very Low | Major | Quiet | Legato |
| 1 | Fast | Very Low | Major | Loud | Staccato |
| 1 | Fast | Very High | Major | Quiet | Legato |
| 1 | Fast | Very High | Major | Middle | Legato |
| 1 | Fast | Low | Major | Quiet | Legato |
| 1 | Fast | Low | Major | Loud | Staccato |
| 1 | Fast | High | Major | Middle | Staccato |
| 1 | Fast | High | Major | Loud | Legato |

Unhealthy

| | | | | | |
|---|---|---|---|---|---|
| 4 | Moderately Slow | Very Low | Minor | Quiet | Staccato |
| 3 | Very Slow | Very Low | Minor | Quiet | Staccato |
| 3 | Slow | Very Low | Minor | Loud | Staccato |
| 3 | Slow | Very High | Minor | Loud | Staccato |
| 2 | Very Slow | Very High | Minor | Quiet | Staccato |
| 2 | Very Slow | Low | Minor | Loud | Staccato |
| 2 | Slow | Very Low | Minor | Quiet | Legato |
| 2 | Slow | Low | Minor | Quiet | Staccato |
| 1 | Very Slow | Very Low | Minor | Quiet | Legato |
| 1 | Very Slow | Very Low | Minor | Loud | Staccato |
| 1 | Very Slow | Very Low | Major | Quiet | Staccato |
| 1 | Very Slow | Very High | Minor | Quiet | Legato |
| 1 | Very Slow | Very High | Major | Loud | Staccato |
| 1 | Very Slow | Low | Minor | Middle | Staccato |
| 1 | Very Slow | Low | Minor | Loud | Legato |
| 1 | Very Slow | High | Major | Quiet | Staccato |
| 1 | Very Fast | Very Low | Minor | Quiet | Staccato |
| 1 | Very Fast | Low | Minor | Quiet | Staccato |
| 1 | Very Fast | Low | Major | Quiet | Staccato |
| 1 | Slow | Very Low | Minor | Quiet | Staccato |
| 1 | Slow | Very Low | Minor | Middle | Legato |
| 1 | Slow | Very High | Minor | Quiet | Staccato |
| 1 | Slow | Very High | Minor | Quiet | Legato |
| 1 | Slow | Very High | Major | Quiet | Legato |
| 1 | Slow | Low | Minor | Quiet | Legato |
| 1 | Slow | Low | Major | Loud | Legato |
| 1 | Slow | High | Minor | Loud | Staccato |
| 1 | Moderately Slow | Very Low | Minor | Loud | Staccato |
| 1 | Moderately Slow | Very Low | Minor | Loud | Legato |
| 1 | Moderately Slow | Very High | Minor | Quiet | Staccato |
| 1 | Moderately Slow | Very High | Minor | Quiet | Legato |
| 1 | Moderately Slow | Very High | Major | Quiet | Legato |
| 1 | Moderately Slow | Very High | Major | Middle | Staccato |
| 1 | Moderately Slow | Low | Major | Loud | Staccato |
| 1 | Moderately Slow | High | Minor | Quiet | Legato |
| 1 | Moderately Fast | Very Low | Minor | Loud | Staccato |
| 1 | Moderately Fast | Very High | Major | Quiet | Staccato |
| 1 | Fast | Very High | Minor | Middle | Staccato |
| 1 | Fast | Very High | Minor | Loud | Staccato |

For both healthy and unhealthy models, the most popular choices agreed along several factors. Aligned with FIG. 9, almost all popular healthy compositions believe the melody should be fast and played in a major key generally in a higher pitch, while the unhealthy composition should be the opposite while also (for the most part) being staccato. However, participants were unable to agree on which dynamic indicated what level of health. While they mostly agreed healthy models should be in a loud or middle dynamic, they could not agree if unhealthy models should use quiet or loud.

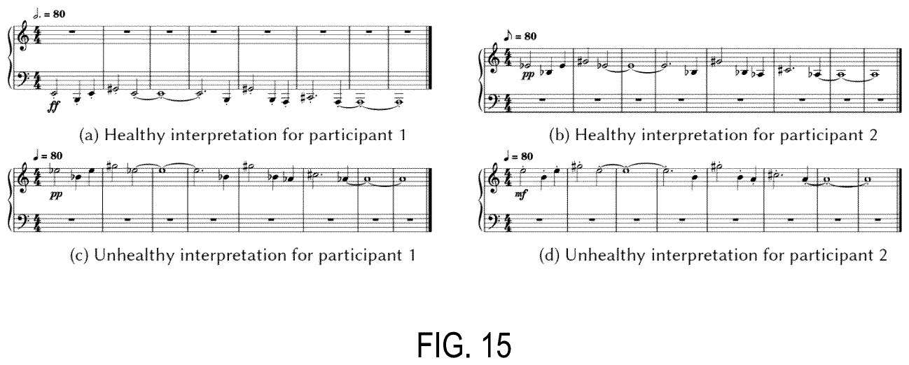

While common trends are observed in individualized music models, the 52 participants generated a large sample of models with 39 unique healthy and unhealthy models. Models differed between participants, indicating participants may have perceived the healthiness of music very differently from others. As an example, FIG. 15 shows the healthy and unhealthy models for two participants. The two disagreed on every feature in the healthy model and only agree on the tempo and pitch of the unhealthy model. The vast number of models indicates few users may abide by every trend but are individualized in their exact preferences.

However, while there was a wide range of preferences for different music features, there were also shared models between participants. In all, almost half of all healthy models were identical to at least another model for a different participant. Only 28 (53.9% of) participants had unique healthy models. Identical unhealthy models were slightly less common, with 31 (59.6% of) participants having unique unhealthy models. Given the number of variables the participants had to choose from, there were a total of 288 unique possible models that could have been designed. It is highly unlikely that the participant pool of 52 users would have so much overlap if each user had completely unique preferences. Thus, while there is still great variation in the personalized models, they are not truly unique to the individual.

Due to the presence of trends, yet strong individuality in the data, the impact of demographics, musical background, and frequency of listening to music on the perceived healthiness of music were further analyzed. Responses to the pleasantness of the music was also compared to the ratings of health in the second survey to determine if the two are related.

All 45 Likert scale questions in the study between different demographic options were analyzed using Kruskal-Wallis tests. Of the 45 tests to detect differences in responses between the genders, one test came back significant (H=5.832, p=0.016). This test indicates female participants perceived the healthiness of physical energy within the all-healthy three-line instrumental melody to be healthier than male participants. Similarly, two tests for race showed significance. Ironically, one of the two tests is for the perceived healthiness of physical energy within the all-unhealthy three-line instrumental melody (H=12.124, p=0.007), which indicates participants that self-identify as Hispanic or Black or African American considered this melody to be healthier than other participants. Another significant test within the race demographic was caused by white participants perceiving the healthiness of the quiet dynamic as lower than participants from other races (H=13.572, p=0.004). Due to the small number of significant tests (2.22%), one can conclude that the discrepancies within the general demographics are due to small sample size or external features, such as personal preferences, that are not accounted for in the study.

As was done with the general demographics, each music demographic was compared across all 45 Likert scale questions to detect any significant differences in responses. Three significant tests were detected across all music demographics. Participants with a voice training background perceived composition U to be less healthy than participants with other music backgrounds (H=10.097, p=0.039). Two tests regarding the frequency with which participants listened to music were also significant. First, participants that listened to music several hours a day and a few hours a month perceived composition E to be less healthy than participants that listened to music a few hours a week or a few hours a day (H=9.657, p=0.022). This result is mildly notable, as participants that listen to very little music and participants that listen to a lot of music provided very similar responses, while participants that listen to moderate music provided a different perception. Lastly, participants that listened to music only a few hours a month perceived the very fast tempo to be less healthy than the other groups (H=8.49, p=0.037). However, once groups with 2 or fewer participants were removed, this test was no longer significant (H=5.018, p=0.081). Thus, one can reach a similar conclusion to the general demographics that the small number of significant tests (1.11%) indicates they are also likely a result of a small sample size rather than actual demographic differences.

Due to the inconsistent and small number of positive tests, one may conclude these demographics did not appear to influence participant interpretations, and that each participant's responses are likely indicative of personal identity traits other than the ones accounted for.

As discussed above, it was theorized that participants may associate healthier music with music they find more pleasing, while unhealthy music could be more associated with unnerving or unpleasant melodies. Therefore, for each melody in the second survey, participants were asked an extra 5-point Likert scale question, assessing how pleasant they found the melody. The Pearson correlation coefficient was used to compare the pleasantness scores to the health scores for each melody. In doing so, a goal was to identify the correlation between the participant's impression of healthiness and pleasantness.

The results show perceived healthiness and perceived pleasantness of a melody are highly correlated. When analyzing all seven single-line melodies, all showed a significant correlation between health score and pleasantness. Both instrumental and piano triple-line melodies also showed a significant correlation between pleasantness and healthiness.

In all, while neither demographics information nor music background show any significant effect on the perceived healthiness of melodies, there exists a strong correlation between how pleasant participants found a melody and how healthy they perceived it. This could provide an avenue for better predicting user responses, as an understanding of what they find pleasant could extend to explain what they find healthy.

The results contain the clear existence of trends explaining how participants perceive the healthiness of music. All five categories of musical features showed a trend, with commonly perceived healthy and unhealthy values. These trends ranged from simple, almost linear trends where healthiness improved as the tempo increased, to more complex, musically ingrained trends, such as more musically common pitches seeming healthier. The strength each feature appears to have on the perceived health level of a melody when they are all combined was also determined. Some features such as tempo are strong indicators of how users will view the music, while features such as smoothness result in no significant difference.

At the most basic level, the presence of these trends indicates users can perceive healthiness through music, showing that the methodology set forth herein is viable. This means health monitoring systems can develop similar methods and deliver information about one's healthiness using music. This may be useful to many fields, including the field of health awareness. Being able to use sonification to present health data creates brand new avenues for users to receive their data. The delivery of this data can become more ubiquitous than ever before, simply requiring the user to be within hearing range of the speaker generating the noise. This can be presented regardless of the user's activities, possibly being developed into calculating the times and activities where the user will be most receptive to the data, rather than waiting for them to interact with a necessary device.

Displaying behavioral health data through music may allow users to better internalize their own health status. It may also aid users in sustained behavior change. Music is well-known and widely accepted for its ability to invoke emotions. Presenting users with their health status through music may trigger emotional connection, spurring them to make healthier decisions than if they were simply told their health information.

To the extent that privacy concerns exist (e.g., due to overlap between user perceptions and the resulting possibility that an unintended listener may be able to partially or fully interpret the user's healthiness), these models may be played at times only the user or people they trust would be able to hear them. This means they could be played at times such as putting on headphones, or perhaps even as an alarm in the morning.

The pleasantness of music appeared to be a strong indicator of the perceived healthiness. This provides a background for music-based information displays and may be used to create simpler music systems. Rather than needing to define a user's exact preferences, it may be possible to simply reduce the user's music tastes to what they find pleasant, or enjoy and infer their health level interpretations from those.

The study identified and showed participants' ability to perceive consistent health levels within three-line melodies. It also showed different instruments are perceived to represent different types of health energy. This means it may be possible to provide multiple types of health information simultaneously.

However, the results indicate participant perceptions may differ between the three-line and single-line melodies. Specifically, when comparing each of the three compositions, participants perceived the piano-based three-lined melody as being healthier than those with a single line. This difference indicates user perception of features' healthiness in a three-line melody may function differently than in a single melodic line. To be practical, three-line melodies may also require their own models to be generated the same way as single-line melodies to better communicate health data.

While the above example utilizes personalized models, there may be other ways to represent multiple kinds of data simultaneously. As seen in the feature trends in FIG. 9, users interpret tempo and pitch through relatively clear scales. It could be possible to use these scales to communicate the data. Rather than combining all five features, the features could be reorganized in attempts to invoke similar, multi-level trends. Then, each musical element could represent a different piece of health information. As an example, suppose someone abides by the commonalities discussed above and finds tempos and pitches to be healthier the faster and higher they get. Rather than using multiple lines, the tempo could simply demonstrate one kind of data, while the pitch represents another. The melody could be played very fast but very low, indicating the health data represented by tempo should be perceived as healthy, while the health data represented by pitch should be viewed as unhealthy.

Additionally, the consistent responses across the three-line melodies show results are unlikely to change as the definition of health becomes more specific. In the instrumental three-lined melodies, each melodic line was narrowed to a more specific type of health (cognitive, emotional, or physical). The tests showed no significant differences between any lines, the overall healthiness of these melodies, and the healthiness of the identical single-line melodies. Thus, one can assume participants' perceptions did not change as health was narrowed to a more specific type.

Through an analysis of the customized music models for each user, the 52 participants created 39 different models for both health and unhealthy perceptions, indicating a wide variety of preferences. Yet, these models still appear to generally follow the trends established when analyzing participants' direct responses. Healthy models predominately utilize faster tempos, while unhealthy models use slower ones.

Upon identifying how and why participant responses fluctuate, these fluctuations could be targeted, thus vastly reducing the number of personal models. For example, the above study identified faster tempos are often healthier, but users' responses sometimes vary between the three fast tempos. Therefore, two fast tempos and two slow tempos could potentially be removed. Users would still be able to perceive healthiness in generally the same way, and there would not be as much variation in the models.

Analyzing the impact of demographics and music background on music healthiness perception portrayed few significant differences in participant responses. While there were the occasional signs of significance, these are likely a signal of small sample size due to their inconsistent appearances. Some demographic groups were very small, making them vulnerable to outlying data. It is possible other demographics, both musical and non-musical may be viable predictors of user responses, but not the ones collected in this study. Should a predictor be found, this could be used to further generalize the data, decreasing the burden on the user to provide data to generate customized models.

The lack of significance in the impact of musical ability and demographics on music healthiness impression is notable, as music psychologists note both can influence music preference. Therefore, one would expect these would also influence the perceptions of how musical features are perceived as healthy or unhealthy. Yet, the above results show no clear trend. This may indicate determining the healthiness of music relies on different existing knowledge than music taste. In some regards, this difference may make sense. Music taste tends to be a relatively intuitive decision. While this will sometimes require thought such as disliking a song due to disagreeing with the theme of the lyrics, it is common for one to decide quickly and easily determine what they like.

There is another possible factor that may have influenced how people perceive healthiness through the music in the study. Bach Cococo's music style is based on that of Johann Sebastian Bach, the famous 18th-century German composer. This means Bach CoCoCo's music, and the study music by extension is based on the western music style, which are musical styles originating from Europe. Western music follows the same set of rules, regardless of how different the melodies sound. As the name implies, non-western music originated outside of Europe and follows different rules depending on the geographical location the music originated from. After becoming accustomed to one style, other music styles may sound strange. While barriers between music styles have been broken over time, they were highly present at the time of Bach, meaning the study music is based on the western music style rather than the more common modern-day combinations of styles. It is possible the methodology above may be modified to include music from non-European cultures, to the extent that ethnicity influences user perceptions.

The methodology above may further be modified to account for the possibility that changes that were attributed to musical traits may, in part, be due to changes in the tune itself. Modified implementations may include multiple melodies, with the same number of lines, containing the same health models to account for this possibility. Additionally, it may be possible to expand the selection of melodies. Moreover, it is possible that user perceptions may change over time. What sounds healthy while a person is at work may not sound healthy to a user relaxing at home, or even in the same context several weeks apart. A modified study may account for this possibility and be designed to assess the impact and viability of portraying health data over time through a longitudinal study. Modified studies can also expand into more complex musical features. The features in the above study were basic but can be applied to essentially any melody. However, there are other musical features that could be included in modified work, such as slurs, chords, accents, crescendos, decrescendos, and vibratos. One example, directed to chords, will be discussed in more detail below.

The above music model generation may be implemented in the form of a mobile application where participants can listen to personalized melodies generated from their real-time biological data. Using IoT devices, such as FitBit and personal phones, one may present a study that collects users' biobehavioral data and creates music representing the healthiness of this data for users to listen to. This may focus on aspects of participants' lives they have direct control over, such as promoting more activity or sleep.

### Activity Level Calculation

The next step in an example of the methodologies herein, after generating a music model, involves establishing a means to transform behavioral data into a corresponding physical activity level. In an example, Fitbit Senses were used to gather data from study participants. The algorithm and study are designed around participants' step data, specifically focusing on healthy daily and hourly step count thresholds recommended by literature and also calculated from an individual's historical data. The combination provides four numeric scores each ranging from 0 to 25 which are subsequently aggregated to generate a unified score between 0 and 100. Once designed, the 0-100 activity score was converted to a 1-5 scale for the five musical wellness levels. To make the algorithm more accurate than just linearly mapping scores, it was decided to base this conversion on the distribution of previously collected step data, corresponding to naturalistic Fitbit data from 158 college students. The algorithm was then used to calculate 13,667 distinct activity scores. The quartiles of these scores were then calculated, to serve as the bounds for each of the five physical activity levels.

The initial segment of the algorithm compared a participant's daily step count to the recommendations provided by the CDC. A score of 0 was assigned if the participant took fewer than 4,000 steps, while a total of 25 points were granted for exceeding 8,000 steps. For step counts falling between 4,000 and 8,000, a proportional score between 0 and 25 was assigned.

The subsequent phase of the algorithm allotted points to participants based on the timing of their steps. Existing research suggests that intermittent walking breaks have favorable effects on both mental and physical health. However, the literature offers limited guidance regarding the optimal timing and duration of these breaks. This portion of the algorithm was patterned after Fitbit's recommendation, which suggests taking 250 steps for 9 hours during the day. Participants earned 2.77 points (calculated as 25 divided by 9) for each hour they achieved this goal, up to a maximum of 25 points.

The third component of the algorithm rewarded participants for surpassing their usual daily step counts. It computed the average and standard deviation of their daily step counts over the past week. Participants received 0 points for falling one standard deviation below the average and a full 25 points for exceeding it. The final component of the algorithm analyzed the timing of participants' steps, promoting consistent schedules that have been linked to improved stress levels and depression. Each day was evaluated using a sliding window of hourly segments, calculating the standard deviation for each hour of the day over the past week. Participants were awarded points for hours with a standard deviation under 500 steps, indicating adherence to a consistent physical activity schedule.

In total, 62 students from an American university were enrolled for this study. Among them, 31 participants identified as female, 30 as male, and 1 as non-binary or third gender. The participants' average age was 22.7 years (σ=3.8 years). Nearly all participants completed the study, with only three exceptions. One participant failed to meet the initial compliance criteria and was consequently excluded, while two participants withdrew during the study-one due to illness and the other for undisclosed reasons. As compensation, participants were allowed to retain their Fitbit devices or receive compensation up to $77. The study was approved by the university's institutional review board.

The 61 participants who passed the compliance period completed 3,441 surveys during the study, including 1,728 music and 1,713 textual surveys. On average, each participant completed approximately 56.41 (σ=10.23) surveys, out of which 28.33 (σ=5.84) were music surveys, and 28.08 (σ=5.90) were textual surveys. Quantitative data and qualitative participant feedback were the focus in conducting the analysis. The quantitative analysis primarily centers on daily step counts recorded by participants' Fitbits and their interpretations of the music in relation to the intended message. Unless stated otherwise, linear mixed models from Python's statsmodels package were used for the statistical analysis. Linear mixed models were chosen because most tests include multiple data samples per participant, violating the random sample assumption of most common statistical tests. All linear mixed models in the analysis account for per-participant differences. On the qualitative front, into participants' responses gathered through the final reflection survey were analyzed. The analysis provides insight into how participants perceived the approach of conveying wellness information through music.

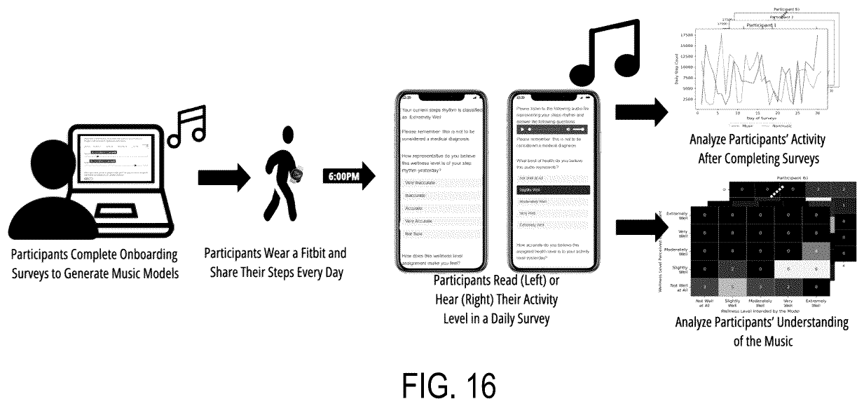

FIG. 16 illustrates the study and approach to quantitative analysis. Participants began by completing onboarding surveys to generate their musical models. They then proceeded to wear a provided Fitbit for upwards of 76 days and complete daily surveys containing feedback on their physical activity. For analysis, it was first sought to identify the relationships between music and physical activity. This analysis seeks to identify whether participants were more active after hearing their melodies, what musical features had the largest impact on activity, and whether baseline activity levels influenced what made music seem healthy. Then, the manner in which the melodies were interpreted was analyzed, for example in view of how frequently participants interpreted music to be the same physical activity level as the model intended to portray and identify contextual factors that influence these interpretations.

Participants averaged 8,700 (σ=3, 635.74) steps a day during the baseline period, 8,704 (σ=3, 376.85) steps after completing a textual survey, and 9,260 (σ=3, 623.52) daily steps after finishing a music survey. Participants were further categorized into groups based on their physical activity level rather than evaluating the entire population to avoid overlooking individual variations. Participants were segmented into three activity-level groups based on the CDC's recommended daily step counts. Participants averaging fewer than 4,000 steps a day during the baseline period were classified as low activity (7 participants), those exceeding 8,000 steps were classified as high activity (31 participants), and individuals falling between 4,000 and 8,000 steps were considered middle activity (23 participants).

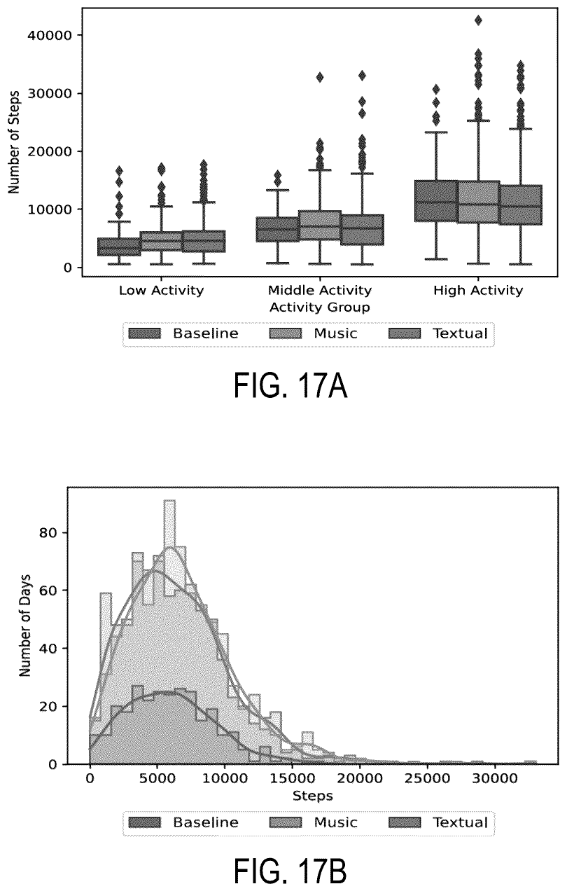

First, changes in participants' daily step counts after receiving each type of feedback were investigated. FIG. 17A and FIG. 17B present data collected regarding daily steps. The baseline was collected during the week where participants received no feedback. music and text contain the steps taken the day after a participant completed each type of survey. The mean and standard deviation of participants' steps are shown in FIG. 17A and Table 10. FIG. 17B shows the frequency of daily step counts taken by low and middle-activity participants during each condition. As can be seen, when ignoring per-participant differences in behavior, the average steps taken by all participants were highest under the musical condition. Looking more closely at the groups, while the combined low- and middle-activity participants also averaged the most steps while receiving musical feedback, the high-activity participants averaged the most under the baseline condition. Middle-activity participants showed increased steps compared to the baseline during both conditions and the most steps during the musical intervention. Low-activity participants took, on average, about a thousand more daily steps during both feedback conditions than the baseline period. However, they took the most steps during the textual feedback. As such, when analyzing the average steps participants took in each activity level group, participants were more active after hearing musical feedback.

TABLE 10
Average Number and Standard Deviation of Daily Steps Taken
by Each Activity Group During Each Condition in the Study

| | Baseline | | Musical Feedback | | Textual Feedback | |
|---|---|---|---|---|---|---|
| Activity Group | Mean | Std Dev | Mean | Std Dev | Mean | Std Dev |
| High | 11,702.88 | 5,306.72 | 11,686.48 | 5,939.21 | 11,160.03 | 5,528.69 |
| Middle | 6,657.23 | 2,950.89 | 7,415.99 | 3,940.29 | 6,929.96 | 4,102.71 |
| Low | 3,950.41 | 3,216.30 | 4,907.41 | 2,930.33 | 5,127.54 | 3,510.77 |
| All 3 | 8,700.17 | 3,635.74 | 9,260.09 | 3,623.52 | 8,704.38 | 3,376.85 |
| Low & Mid | 5,945.44 | 3,211.96 | 6,786.88 | 3,926.16 | 6,440.39 | 4,025.55 |

While it can be seen that middle-activity participants appeared to benefit the most from the musical feedback, it remains unclear why this group in particular would benefit the most. Intuitively, one would expect low-activity participants to most benefit, as they can improve the most. Analyzing the differences between the groups, the musical models for the middle-activity participants varied the intended wellness level more frequently than the models for low and high-activity participants. In other words, changes in a middle-activity participants' level of physical activity were more likely to be reflected by the musical models. For low-activity participants, the music communicated the same two wellness levels (“slightly well” and “moderately well”) in 71.49% of melodies, indicating while the musical feedback did make these participants more active, the melodies were largely consistent throughout the entire study. Meanwhile, the high-activity participants received the wellness levels of “very well” and “extremely well” in 79.66% of melodies. These participants were already physically active and were likely to appreciate a well-composed melody without any major changes to their daily routine. However, the participants with moderate activity levels frequently heard melodies with different wellness levels, with each level appearing between 16.79% and 31.03% of melodies. Because the daily routines of the moderate activity participants were neither exceptionally healthy nor unhealthy to begin with, the changes in their daily behavior were better highlighted in the melodies. This potentially encouraged them to become more active, as they could hear the differences their behavior made in the music.

The manner in which participants' steps varied during the different conditions when accounting for personal differences in behavior was also analyzed. Using linear mixed models, each participant's steps during the baseline were first compared to those taken during their first feedback method. Given that participants' activity during the second condition may be influenced by their first month of feedback, only a participant's activity during the first condition was compared to their baseline steps. Although non-significant, participants took more steps after hearing musical feedback than the baseline, regardless of their baseline activity level (p_{All}=0.091, p_{High}=0.077, p_{Middle}=0.309, p_{Low}=0.106). Similarly, high-activity participants took more steps during the textual feedback condition than the baseline (p=0.811). However, the models indicate many participants were actually less active after receiving textual feedback than during their baseline period (p_{All}=0.859, p_{Middle}=0.672, p_{Low}=0.758). Although non-significant, these models further support the initial finding that participants were more active while receiving musical feedback compared to their baseline activity.

Finally, the two feedback types were compared to each other. It was initially observed that participants were significantly more active during the second feedback condition (p=0.047), regardless of the order of conditions. This could indicate that the first condition influenced the second, or, since the study was run during the spring semester, participants were more active due to the improved weather. Due to this effect, the models to were separated to consider participants based on the feedback they received first, generating linear mixed models for just the participants who received the music first, and another for those who received the text first. In line with the aforementioned effect, participants who started with the musical feedback showed a non-significant increase during the textual feedback (p_{All}=0.361, p_{High}=0.861, p_{Middle}=0.173, p_{Low}=0.505), indicating they may have marginally benefited from the textual feedback over the musical feedback. The participants who received the textual feedback first, however, showed a significant improvement during the musical feedback (p_{All}=0.000, p_{High}=0.015, p_{Middle}=0.002, p_{Low}=0.000). When considering that the models indicating textual feedback worked better were non-significant while those supporting musical feedback were significant, one can conclude that participants were generally more active during the musical feedback compared to textual.

Next, the correlation between musical features and participants' physical activity was determined. Information regarding the musical features is set forth in Table 2 above. This determination began by investigating a potential relationship between a participant's average activity and their baseline perceptions of musical wellness. This analysis could provide insights allowing for a small level of personalization without burdening the user to create the musical models explicitly. Each participant's well and unwell choices for each musical feature from onboarding were collected and ordinal regression was used to analyze whether the participant's average steps during the baseline week could indicate the participant's choices. Participants' baseline activity appears to relate to one musical feature. While more active participants perceived faster melodies to be healthier, participants who took fewer steps during the baseline condition were more likely to perceive slower tempos as healthier (p=0.040). This indicates a small level of personalization could be inferred from participant behavior, rather than explicitly their preference.

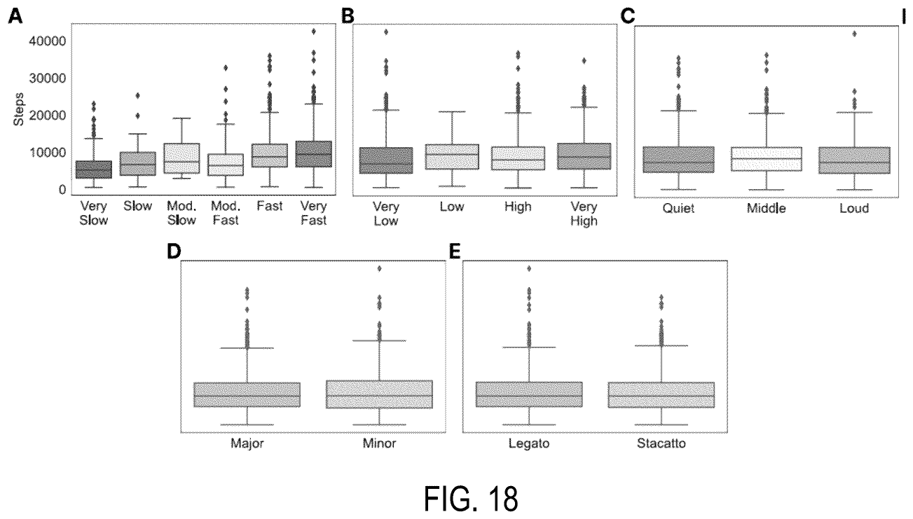

Since participants' perceptions of musical wellness correlated to their baseline activity, relationships between musical feature values were investigated by analyzing the number of steps participants took after hearing melodies with the various features. The distribution of steps taken the day after hearing every value for each musical feature is shown in FIG. 18, in which graph (A) shows results for tempo, graph (B) shows results for pitch, graph (C) shows results for dynamics, graph (D) shows results for key, and graph (E) shows results for smoothness. Although non-significant, the models indicated potential trends. Specifically, participants were more active after hearing faster tempos (p=0.096), higher pitches (p=0.547), quieter dynamics (p=0.597), minor keys (p=0.747), and smoother melodies (p=0.224). The different activity level groups were then investigated. All three groups appeared to be most active after hearing a different type of feature. High-activity participants' steps most correlated with the song's key (p=0.083) and low-activity with pitch (p=0.205). Meanwhile, middle-activity participants significantly correlated with the song's tempo (p=0.027).

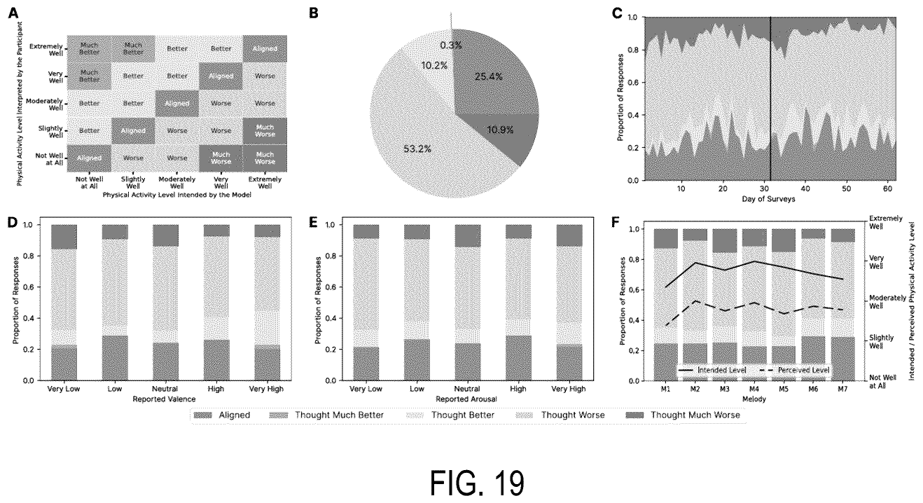

Further analysis was conducted to determine how participants interpreted the melodies' messages built from their activity data, and how in-the-wild contexts influenced their interpretation. Throughout this analysis, the focus was on a phenomenon referred to as “alignment.” Alignment is defined as the relationship between the physical activity level interpreted by the participant compared to the activity level the model intended to communicate. If the participant interpreted the melody to contain the same activity level the model was trying to communicate, then the user and model are considered to be “aligned.” FIG. 19 illustrates the alignment findings from the study. Graph (A) of FIG. 19 presents a summary of how alignment terms are defined. The term “thought better” indicates the participant interpreted the music sounded healthier than the model intended, while “thought worse” means the opposite. Lastly, the terms “much better” and “much worse” are used to indicate this misalignment was off by at least two physical activity levels.

Next, the distribution of survey responses across different categories was analyzed. Graph (B) of FIG. 19 shows the overall proportion of music survey responses falling into each alignment category. Participants' interpretations and the model's intended message were aligned in just 25.41% of survey responses, slightly above the chance alignment rate of 20%. Two-thirds of responses (64.06%) interpreted the melody as conveying a lower activity level than intended. Conversely, only 10.53% of responses reflected an interpretation of the music as better than intended. This negative skew in participants' interpretations of musical feedback will be discussed below along with the potential influence of algorithm design on music melody construction.

Given the subjective nature of musical feedback, participants' interpretations can evolve over time, necessitating a “recalibration” of the musical models. To assess this temporal evolution, the distribution of musical survey responses was tracked across alignment categories for each study day. Graph (C) of FIG. 19 shows the proportion of responses falling into each type alignment grouping for each day of the study. Using linear regression, changes in the proportion of responses were examined for each of the five alignment categories. The analysis reveals that, as the study progressed, participants became more inclined to interpret the music as better than the model intended (β=0.001, p=0.014), while their likelihood of perceiving the music as much worse than intended decreased (β=−0.002, p<0.001). In contrast, the remaining three alignment categories exhibited no significant changes over time (p_{Aligned}=0.105, p_{Much Better}=0.688, p_{Worse}=0.752).

Next, the manner in which participants' emotional state affected how they interpreted the melodies was investigated. The alignment between participants' activity interpretation of melodies and the model's intended activity level was analyzed while considering participant's self-reported valence and arousal from each survey. The impact of valence on alignment was first analyzed on alignment was first analyzed using a linear mixed model, yielding a significant result (p=0.002) Graph (D) of FIG. 19 shows alignment sorted by self-reported valence. Graph (E) of FIG. 19 shows alignment sorted by self-reported arousal. As shown in FIG. 19 graph (D), participants tended to interpret the melody as better than intended when their valence was higher. In other words, participants experiencing positive emotions were more inclined to interpret the presented melodies as containing positive feedback than those experiencing negative emotions. Next, the influence of arousal on how the melodies were interpreted was investigated. The model revealed that participants' interpretations did not significantly vary with arousal (p=0.428). Consequently, as reflected in arousal, the intensity of emotions did not significantly affect how the melodies were perceived.

These findings suggest that a user's positive or negative mood can alter their interpretation of melodies, with positive emotions leading to more favorable interpretations. However, the intensity of the emotion, as measured by arousal, does not seem to play a significant role in these interpretations. As set forth herein, musical feedback systems may either mitigate the influence of valence on users' interpretations or incorporate the user's current valence when generating music.

To maintain long-term engagement with the system and enhance compliance, it is beneficial to introduce variability into the underlying songs of the melodies. This approach may keep the system interesting for users over an extended period. To address this, the study was structured so that each day of the week featured a different song while maintaining consistent alterations in musical features to convey physical activity levels. The technique of embedding wellness data in music can apply uniformly across different songs. Graph (F) of FIG. 19 shows alignment for each of the 7 songs that feedback was embedded in.

The linear mixed model revealed no significant difference among the seven melodic tunes (p=0.073). However, as illustrated in graph (F) of FIGS. 19, M6, and M7 appeared to have higher correct alignment than the other five (p=0.000). Interestingly, these songs were played on Saturday and Sunday, respectively. There were no differences between weekday melodies (p=0.074) and weekend melodies (p=0.926). While the difference in interpretation might be attributed to the choice of melodies, an alternative explanation could be that participants might have been in different environments or exhibited distinct behaviors during the weekend. An initial hypothesis was that participants' weekend moods could differ, yet linear mixed models on self-reported valence (p=0.122) and arousal (p=0.703) showed no significant disparities between weekdays and weekends.

A subsequent hypothesis considered that participants' interpretations remained consistent, but the activity levels intended by the model varied on weekends. Each day's average intended and interpreted messages were calculated, as shown in graph (F) of FIG. 19. Linear mixed models indicated a correlation between weekends and lower intended activity levels (p=0.003), yet no significant difference in the activity levels participants interpreted (p=0.218). These tests support the final hypothesis that the shift in alignment during the weekend corresponds to a change in participants' physical activity rather than their interpretations.

Upon concluding the study, each participant was invited to provide feedback through a final reflection survey. 55 of the 59 invited participants completed the survey. This survey posed a series of questions to capture participants' experiences during the study. Unlike the description above, which primarily centered on behavioral data and daily survey responses, the analysis here will exclusively concentrate on the insights gleaned from the responses to this concluding survey. The survey commenced by prompting participants to express their level of agreement with a series of statements using a Likert scale.

Subsequently, participants were presented with three open-ended questions that delved into their experiences with the melodies, such as how the melodies contributed to their motivation, what aspects they found appealing, and any aspects they found less appealing about this approach. Due to the small sample size, the qualitative responses were analyzed with manual encoding. To do so, all responses were placed in a spreadsheet, reviewed, summarized, and then grouped based on similar themes. After all groupings were made, the exact text of each review was reanalyzed to ensure it was placed in the appropriate group. This last step was repeated until an iteration when no responses were recategorized. The classifications were then assessed and confirmed.

Participants responded to whether hearing the melody helped motivate them to be more conscious of their physical activity than reading their activity level using a 5-point Likert scale. In total, 58% of participants either agreed or strongly agreed that the melodies played a role in motivating them. However, only 22% indicated that the melodies were more motivating than simply reading their activity levels.

Of the 55 participants, 30 (54%) reflected on how the melodies contributed to their motivation to be more conscious of their physical activity. Among the responses, 13 (24%) participants mentioned that the melodies motivated them by either avoiding “bad” sounding melodies or seeking out “good” melodies. For example, P21 stated, “Hearing a more sad melody motivated me to move around more. Over the study, I began walking more to get happier results.” Five (9%) participants directly referenced specific musical features within the melodies as motivational factors. Some commented on the tempo, with three (5%) mentioning it, while one (2%) each mentioned pitch and key. These findings gather support for the findings above, where weak, non-significant correlations were found between the musical features, with the strongest difference being present after hearing fast tempos. Between the qualitative and quantitative analysis, the results appear to indicate tempo had the strongest relationship with how participants' activity would differ after hearing a melody. Meanwhile, two (4%) participants indicated that the open-ended nature of the melodies made them contemplate the intended activity level, keeping them engaged and curious. For instance, P10 said, “It kept me guessing what my result actually was.” These views lend credence to the underlying theory that musical feedback's open-ended nature would encourage the participants to reflect on their activity.

On the other hand, four participants (7%) expressed that the melodies did not inspire them. P41 stated, “Honestly, the melodies were highly confusing, and if you're having a bad day, playing bad music to it just makes your mood worse.” Some responses didn't fit into the mentioned categories. P30 responded about the study, indicating that it made them more mindful of their sleep and activity levels. P47, dealing with chronic health issues during the study, expressed uncertainty about whether the melodies helped them. While the melodies motivated many participants, modified approaches may consider combining music and textual representations to cater to a broader range of preferences and motivations.

According to a 5-point Likert scale, 78% of participants reported enjoying hearing their activity levels through music melodies, while 14% disagreed, and 5% neither agreed nor disagreed. Only one participant (2%) strongly disagreed. These results indicate strong overall support for the concept of musical feedback for physical activity. Given that more than three-quarters of participants enjoyed it, and the majority had positive responses, these findings suggest that musical feedback for physical activity could be considered a viable option for promoting healthier behaviors.

Participants were also asked to explain what they liked about hearing melodies that encode their activity level information. All 55 responses answered this question. 21% of respondents (12 participants) expressed their enjoyment of the novelty surrounding the melodies and the overall concept. They articulated sentiments such as “Music wellness interpretations provide a holistic approach to health and well-being” (P7). Similarly, 12 participants (21%) highlighted their appreciation for the engaging and creative qualities of the melodies. They expressed that the music-based activity interpretations were more captivating and enjoyable than text-based representations. P28 remarked, “I felt like the music wellness interpretations were more interesting and fun than the text representation.” Approximately 14% of participants commented on enjoying the melodies themselves. They relished the opportunity to listen to a brief melody each day, especially when they were aware of their high level of physical activity from the previous day. For example, P25 mentioned, “I liked getting to listen to a little melody every day, especially when I knew that I was very active the day before.” It is important to note that if participants find the melodies to be fun and engaging, it could mean that musical feedback has the potential to keep users interested over time. This is particularly important for systems that aim to promote behavior change. Another 8 (14%) noted that they derived pleasure from the emotional impact of the melodies. They appreciated that the melodies sounded cheerful on days when they were active and healthy, finding it to be a somewhat rewarding and mood-enhancing experience. P52 stated, “I liked that when I was being active and healthy, they sounded cheery. It was somewhat rewarding and made me happy.” Participants noting the emotionality of the melodies is an additional sign that the musical feedback may encourage intrinsic motivation, as the participants will be driven to receive melodies that make them feel better about the feedback. Lastly, 5 participants (9%) expressed satisfaction with the melodies' personalization: “I liked how personalized they were. It was nice” (P34).

The remaining responses were categorized into four less common themes. Two participants (3%) expressed their satisfaction with the simplicity of understanding the melodies, with comments like, “I could instantly recognize the wellness level without having to think back on my day” (P4). Another 2 participants (3%) discussed the privacy aspect of the melodies, though their viewpoints contradicted each other. P41 noted, “They were very creative from a privacy perspective: Other onlookers likely had no idea what information was being communicated,” while P44 mentioned, “I feel like if I would have shared the melodies with a friend, they would be able to understand what my health levels were.” An additional 2 participants (3%) provided negative feedback about the melodies, offering concise comments such as, “I did not like them” (P5). The remaining four responses (7%) were considered off-topic. Among these, 3 participants (5%) commented positively on the wellness algorithm, with P8 stating, “I liked the measurement of my wellness.” The final respondent, P1, (2%) expressed enjoyment in the reflective nature of the daily surveys, noting their pleasure in “daily [evaluation] of thyself while [filling out] the survey end of the day.”

A significant number, comprising 51% of the participants, found it challenging to interpret the melodies, often struggling to discern the intended physical activity level conveyed by the music: “It was sometimes challenging to figure out what the wellness level represented by the music was intended to be” (P55). Some participants preferred a more varied and fuller sound than the basic piano tones used in the melodies, constituting 16% of the responses. Another 9% mentioned difficulties in remembering the specific musical features they had associated with each activity level, which occasionally led to guesswork. This awareness matches the results of the quantitative analysis above, participants struggled to interpret the exact message from the music. These reports, however, highlight that participants knew they were misaligned with the system. Implementations could utilize this knowledge to receive feedback and correct the models until participants and the model can correctly communicate.

Some (11% of) participants reported not enjoying the emotional impact of the melodies, particularly when slower tempos were used, such as P13 saying, “It upset me to have slow tempos.” Other participants, constituting 5% of the responses, expressed dissatisfaction with the music's open-ended nature. P41 summarizes this viewpoint, “I have to actively listen and interpret the music, and for health situations I have no desire to interpret what a doctor is telling me. I want them to just tell me so I can fix it as soon as possible.” Two participants (3%) compared the musical interpretations and a textual format, with P24 commenting “It felt too vague and open to interpretation, I was never completely sure if I did well or not. I prefer the written feedback, as it is inarguable and quick and simple to interpret.” P10, however, suggested the simultaneous use of both musical and textual formats. P16 (2%) voiced privacy concerns, stating discomfort with the idea that other people could hear the melodies. The feedback received suggests that although musical feedback could motivate some users to be more active, it should not be mandatory. As everyone has different preferences, some may prefer more straightforward approaches, while others may prefer the more traditional vision-based feedback.

Finally, some participants expressed off-topic concerns about the study, rather than the music itself. 3 participants (5%) focused on the algorithm's accuracy concerns, such as receiving unsettling tones after achieving certain activity levels or that the melodies were too finely categorized, conveying a sense of granularity. P14 (2%) indicated their sole complaint was related to the timing of the surveys, expressing dissatisfaction with the time of day the surveys were sent.

### Results Analysis

Among the benefits provided by systems and methods according to the present disclosure is the use of musical feedback to encourage users to be more active using their actual physical activity data. While comparative models have been proposed, applying them to encourage real-world behavior demonstrates the possibility of using music as a ubiquitous tool for delivering wellness feedback. Such ubiquitous delivery means musical systems could be used to communicate with the user without requiring them to be actively engaged with a device, potentially creating brand new ways and times to communicate with the user that might be meaningful, such as an alarm clock. The present system is believed to be the first to experiment with delivering personalized behavioral feedback rather than in-the-moment sonification of a live signal.

Comparative systems exist to encourage physical activity through creative approaches. Several such systems will be briefly discussed to highlight how the findings herein relate to and differ from these other studies. This study used a longitudinal approach and equipped every participant with their own Fitbit, providing a level of resolution in participants' activity that not every comparative study reports. Other comparative studies evaluate their results using metrics such as number of activity goals met or qualitative reports. As such, direct comparisons of efficacy may be challenging, but it is still possible to compare the extraneous findings from each study, particularly a common trend of using creative feedback to foster intrinsic and extrinsic motivation for the purported benefits of each system.

The studies described herein demonstrate the benefits of creatively presenting feedback on physical activity through intrinsic motivation, fostering reflection and emotionality. Reflection Companion encourages reflection through dialogue with a conversational agent. In the above qualitative analysis, it was found that musical feedback has the potential to encourage reflection in users. This is because the message conveyed through music requires active interpretation by the user, as opposed to being told directly. Similarly, these systems also promote reflection by requiring users to reflect on their actions, which in turn encourages them to be more active. Some comparative approaches motivate participants by eliciting specific emotions, like in WhoIsZuki, where only those who meet their physical activity goals can help the main character find his brother. According to feedback from some of the participants herein, listening to certain melodies affected their emotions. This suggests that musical feedback could encourage engagement as people tend to be more active when they hear happier melodies. In this way, musical feedback is in line with research that has shown how creative communication can effectively promote wellness information and stimulate reflection and emotional engagement.

Another trend within creative approaches to physical activity feedback is generating a sense of community for extrinsic motivation. Gamified approaches often encourage users to compete with one another. StoryMap, as a comparative example, encourages community members to share their stories, and 3D printed wellness artifacts sparked conversation and competition between participants. Each of these systems, either intentionally or incidentally, fosters a sense of community around the feedback. Communities can serve as support networks, connecting individuals with similar goals to encourage physical activity. The present study does not capture this aspect of creative feedback. To avoid any potential issues in this study, efforts were made to ensure that participants did not communicate with each other. Although some participants may have recognized each other, this aspect of the data was not specifically investigated. However, it is to be noted that one participant in the qualitative analysis mentioned that they felt comfortable sharing the melodies with their friends. This could indicate, much like the 3D printed wellness artifacts, that with proper configuration, the artistic nature of the melodies could potentially foster community. If participants enjoyed listening enough, they could share the tunes with friends and family, creating a sense of community around the feedback.

Although creative wellness feedback may foster community, engagement and reflection, other feedback approaches still provide some benefits over creative feedback. Sometimes, direct interventions can be helpful for people who do not have enough knowledge on how to be physically active. In such cases, these interventions can provide necessary information to help users make a change, rather than relying on self-reflection to feel motivated and engaged. The creative and direct forms of feedback need not be mutually exclusive. Musical feedback can be used in conjunction with more straightforward reflection to provide both direct feedback and emotional data, thus combining the benefits of both types of systems.

The findings above imply that music can serve as an effective feedback mechanism to motivate some participants to increase their physical activity. This could be integrated into health apps or systems for a more engaging user experience. The study showed that musical feedback improved behavior slightly over the baseline, and significantly over textual feedback. However, depending on their average activity, participants responded differently to different musical features. Thus, the algorithm may factor in user-specific behaviors and preferences. Besides, while there is a clear preference for rewarding users with more pleasant melodies as a response to increased activity, less pleasant-sounding melodies can also serve as effective motivators. These less pleasant melodies can act as warning alarms, drawing users' attention to their inactivity and encouraging them to take action. Implementations can account for the type of feedback the developers want to use. Emphasizing negative-sounding melodies may encourage users to push themselves harder, while positive-sounding melodies may be more rewarding.

Enhancing the melodies for users' enjoyment and engagement could be achieved by considering individual music tastes rather than individual perceptions of musical wellness. The particular approach described above generates all feedback in the classical musical style of Bach. However, given that the participants' average age indicates that less than 3% of the music they listen to is classical, expanding the modeling to encompass a broader range of musical styles may result in melodies that are more pleasant to users, potentially making them sound healthier. Implementations could automatically determine the style of music to deliver by analyzing users' music streaming history, such as on Spotify. This expansion into various musical genres can add appeal to the songs, rendering them more captivating, relatable, and personal to users. Furthermore, factoring in participants' preferred musical styles might even obviate the necessity for specific musical features. For instance, a model could utilize songs aligned with an individual's musical taste to convey healthy behavior while employing songs the user dislikes to represent unhealthy behavior.

Moreover, this study delves into musical feedback as a distinct form of physical activity feedback. Nevertheless, in practical implementation, the musical delivery of feedback could be seamlessly integrated with vision-based approaches such as ambient displays, textual and graphical representations, or gamified systems. This musical and visual feedback integration could create a synergistic relationship where each form complements the other's strengths. Incorporating musical feedback could aid visual feedback in conveying the emotional aspect of the message, enhancing overall communication. Simultaneously, visual feedback can mitigate the risk of misalignment in open-ended musical delivery, ensuring that users receive a more coherent and effective physical activity message.

As discussed above, only tempo showed any significant correlation with the steps participants took after hearing a melody. While this indicates tempo may be able to directly influence the inspiration middle-activity users take from the music, it indicates the other features may not. Therefore, it is conceivable that participants do not consider the musical features at all, and future approaches may not need to rely upon them. Music inherently conveys emotions that transcend socio-cultural boundaries. Embedding wellness data in the emotional quality of a song could make the feedback easier to interpret. Incorporating wellness data into the emotional elements of a song could make it easier to understand the feedback.

Additionally, enhancing the emotional connection could improve the effectiveness of musical interventions. Instead of needing to analyze musical features, users would simply reflect on the emotion of the song. While this alteration might introduce a degree of subjectivity into the approach, it could also simplify the interpretation and internalization of the wellness message. Additionally, in light of the overall low alignment, it is also plausible that the utilization of multiple musical features unnecessarily complicates the model. Implementations might explore the possibility of relying on a single feature to convey wellness feedback effectively. For instance, a melody could embed wellness data using solely tempo, which, in addition to encouraging users to be more active, improves the perceived wellness of music. In this scenario, faster melodies could signify the participant performing well as positive reinforcement and encouraging continued physical activity. With this approach, users would only need to interpret the speed of the music rather than consider all five features. This streamlined approach will likely simplify comprehending the melodies and potentially enhance overall alignment. Designing systems around a single factor, such as emotionality or tempo, will make the music easier for users to understand while seemingly remaining effective in promoting physical activity.

Above, it was noted that participants' interpretations of the music became more aligned over time. This may signify a growing familiarity with the model. Initially, at the outset of the study, participants may hear the melodies and perceive them as unpleasant, immediately associating this with unwell data. However, as they listen to the music over several days, participants better understand how the different levels of physical activity sound in the melodies. Consequently, this gradual acclimation refines their perceptions, bringing them closer to the intended message. Thus, practical implementations may to account for this “learning curve” in interpreting melodies or devise strategies to expedite the calibration period. This calibration period can be eliminated by blending music and textual feedback to convey the message clearly while retaining emotional impact.

Practical implementations may be designed to account for potential calibration changes and lowered usage rates with prolonged use. As users become more familiar with the melodies, they may become less novel. This may result in weaker compliance from the users. It is possible that this effect could be mitigated by changing the underlying melody while consistently applying the same musical models to embed the message in the music. However, implementations may account for the possibility that the time required to calibrate may simultaneously make users less interested in the musical feedback as the novelty effect wears off.

The above findings show that the way melodies are perceived can be influenced by various contextual factors. The linear mixed models indicated that the time elapsed since generating the models, the participants' valence and the day of the week significantly relates to how the melody is interpreted. Allowing the model the flexibility to adapt its behavior based on these contextual factors should enhance the consistency of alignment between musical feedback and users' interpretations, ultimately leading to the improved overall performance of the musical feedback system. Context-aware computing may be used to improve music recommendation systems, as building models to account for contextual factors may improve the performance of this system. Context awareness is also possible for addressing privacy concerns, especially if musical feedback is audible to others. As noted above, some users were conscious of the music's potential lack of privacy, and may hesitate to use the system if their wellness data is not adequately protected. Improved context-awareness can facilitate just-in-time active interventions by learning the user's preferred time and setting for receiving musical feedback, such as when they return home from work, and identifying the most receptive time for delivering a message.

Using musical feedback for conveying health information can enhance the accessibility of personal wellness technology by expanding the means of communication beyond visual displays. Developing these systems with simplicity in mind may ensure they are accessible. The analysis above suggests that one approach to case the generation of musical models is to generalize musical features. baseline activity was observed to correlate with participants' perception of wellness from a melody's tempo, which could be exploited to drastically reduce the burden of generating individualized musical models. Through this reduction of mental burden, the case of using the system is improved, making it more accessible to all users.

However, simplifying these models may raise privacy concerns. Given that auditory output is pervasive and can be heard by anyone nearby (if used without headsets), there is a risk that others nearby may perceive and interpret the wellness messages intended for the user. Therefore, secure yet meaningful methods of delivering melodies may be incorporated. For instance, one potential approach could involve playing wellness melodies as the user's morning alarm clock in a setting with minimal unintended listeners. Users may wish to share their data, and doing so can encourage them to be even more active. Applying this finding to musical feedback, it is possible that some users may not be worried about privacy, but rather want their musical feedback to be heard by friends and loved ones. Practical implementations may consider the appropriate deployment settings to ensure the melodies remain accessible, beneficial, and private while avoiding unintended disclosure of personal information.

Thus, the systems and methods examined in the above studies be subject to a number of modifications. For example, it was discovered that middle activity participants benefited the most from the musical intervention. However, no clear demographic or background indicators were explicitly established that could explain why they were more receptive to the musical feedback. Therefore, modifications can focus on defining the target population that can benefit the most from musical feedback. Additionally, the study hinted that musical feedback could be an effective tool for accessibility purposes, especially for visually impaired users. The auditory nature of musical feedback can communicate better with visually impaired users than vision-based approaches. This application of musical models can modified to tailor the models the visually impaired users. Lastly, the study found that, over time, participants' interpretations became better aligned with the model, which may indicate that they learned how to understand the feedback. The implementation of musical models remained consistent, allowing participants to learn how to interpret the feedback. However, modifications could use an adaptive algorithm to implement musical feedback. This approach will allow the system to learn how to communicate with users while they simultaneously learn how to interpret the feedback.

### Modification Example: Chords

In one example, the methodologies discussed herein may utilize chords in addition to the five features described above (tempo, pitch, key, dynamics, and smoothness). In music, a note is the most basic element, which consists of a sound at a specified tone held for a specified length of time. A chord, on the other hand, is a group of multiple notes played simultaneously. The triad is the most common type of chord, consisting of three notes, each a small step above the previous. The chord is named after its root, which is the lowest tone. For instance, a chord with the root of C would be called a C-chord. Although triads are the most common, composers can modify triads to produce different sounds. There are many ways to alter a chord, but for this discussion, two will be the focus: tetrads and changing the mode of chords to major or minor. Tetrads are chords with four stacked notes rather than three, while changing the mode of a chord alters its notes, making minor chords sound sadder. A series of chords played in sequence is called a chord progression. Typically, chord progressions comprise the harmony of the song, which serves as the supporting musical voice within a song, complementing and adding to the main voice, called the melody. Throughout this description, the terms chord progression and harmony are used interchangeably.

Progressions are influenced by the key, which specifies the exact notes to be played in each chord. Like chords, keys are named after notes. When the root of a chord is the same as the name of the key, the chord called the tonic. For instance, in the key of C, the tonic is the C-chord. Chords within a key have specific relationships with each other. These relationships are denoted using Roman numerals representing the number of notes away from the chord's tonic root. In the key of C, the chords are represented as follows: C=I, D=ii, E=iii, F=IV, G=V, A=vi, B=vii. Capitalized Roman numerals indicates that the chord will sound major in this key, while lowercase numerals indicate that the chord will sound minor. Regardless of the key, the chords in positions I, IV, and V will always sound major, while the others will sound minor. These chords have special names and properties when transitioning between them. The chord in position IV is called the “sub-dominant,” and a transition from I to IV is considered weakly resolved, which means that the listener may feel like the progression could continue or end. On the other hand, the chord in the V position is called the “dominant,” and a transition from I to V is considered strongly resolved, allowing the listener to feel like the progression has concluded.

Chord progressions impact a song's valence and arousal, with a stronger association with valence. Arousal levels vary with less resolved chords, major keys, higher pitch, and tetrads. Moreover, major keys and higher pitches are linked to higher valence, whereas tetrads are associated with lower valence. It has also been found that ascending chord progressions have positive valence, while descending progressions have negative valence. Nevertheless, comparative studies have not adequately considered the influence of an individual's musical background. One study discovered that musical novices and experts differ in their perceptions of meta-features such as tension and interest. However, another study showed that most musical features are perceived similarly by individuals with and without musical backgrounds. Although these studies suggest that individuals with varying levels of musical background may perceive emotions differently, they do not focus on the emotional tone of the music, such as whether it sounds happy or sad. Some studies investigate connections between musical background and the perceived emotionality of music, although they focus on music as a whole, rather than chord progressions. There exists a further need for systems that may leverage how musical harmony can be used to communicate specific emotions.

In support of this modification, two further studies were conducted. The first study utilized a structured survey to examine the influence of different progressions on participants' emotional perceptions of harmony. A total of 40 individuals were recruited through word of mouth, and 35 of them successfully completed the survey. All participants who completed the survey were included in the analysis. When asked “do you have any formal music education,” 11 participants self-reported that they did not, while the other 24 had experience playing an instrument, could read music, received voice training, or knew music theory. 25 participants were between 18 and 22 years old, 5 were between 22 and 26, and the remaining 5 were over 40 years old.

In this initial study, an aim was to investigate how different types of chord progressions influence perceived emotions and whether these perceptions differ based on a participant's musical background. Specifically, two research questions were presented: RQ1—Do participants with different musical backgrounds perceive the progressions differently? RQ2—Are participants with more musical backgrounds' perceptions of musical emotionality more consistently positive or negative? First differences between the progressions at the population level are analyzed, and then whether musical and non-musical participants differ from population trends or each other.

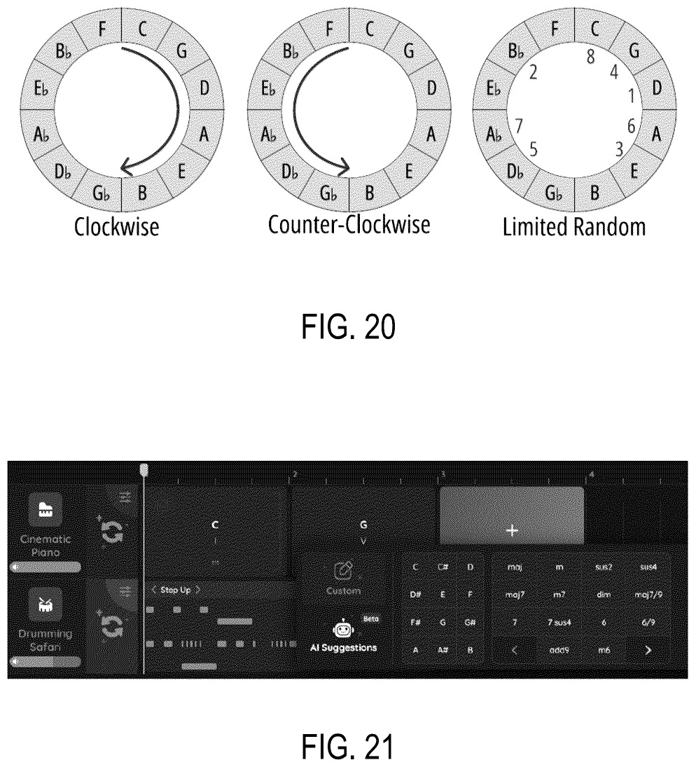

A set of four-chord progressions were designed for participants to listen to and rate based on the perceived emotionality of the harmony. The first progression consists of a change from tonic to dominant (I-V), which is considered strongly resolved and should sound pleasing to the listener. Consequently, this transition also moves the chords around the Circle of Fifths, a musical tool for cyclically changing keys. Since this progression rotates clockwise around the Circle of Fifths, it is referred to as the “Clockwise” condition. The second progression shifts between the tonic and the sub-dominant (I-IV), which still sounds resolved albeit not as concluded as the transition from tonic to dominant. This transition is equivalent to traversing the Circle of Fifths counter-clockwise and is called the “Counter-Clockwise” condition. The remaining progressions do not follow a pattern as structured as the Circle of Fifths. Rather, they are both deliberately designed to sound unresolved ensuring that the chord does not transition to its sub-dominant or dominant. To create a sequence of chords, random numbers were assigned to each of the twelve chords and a random number generator was used to generate the progression. The first progression, which is referred to as the “Limited Random” condition, was restricted to only major triads, despite the sequence being random. The clockwise, counter-clockwise, and limited random conditions are shown on the Circle of Fifths in FIG. 20. The final progression was also randomly generated, although it was not limited to major triads, allowing for a wider range of harmonies. This was identified as the “Fully Random” condition.

To keep the harmony short, each progression was limited to 8 chords, exported to an mp3 file, and placed into a Qualtrics Survey. The survey commenced by asking the participants about their music education level, through the same question noted above with regard to music modeling studies. Then, the participants listened to each of the four progressions in a random order. After listening to each harmony, they were asked to how well four terms apply to the harmony on a scale of 1 (strongly disagree) to 7 (strongly agree).

Ratings were collected for the words “happy,” “tiring,” “satisfying,” and “pleasing.” While most terms were chosen using Russell's circumplex model of affect (better known as the valence-arousal scale), emotional terms are used rather than valence-arousal scales to better study how specific emotions can be invoked. Although each term was analyzed, a focus was on “happy,” due to the aforementioned concept of playing happy music to reward users for positive wellness. Happiness is considered high valence but relatively neutral arousal. The term “tired,” a term with very low arousal but neutral valence, was then added to the study in order to better observe the differences between arousal and valence. Using the circumplex model, the term “satisfying” was identified as falling halfway between “happy” and “tired,” and was included to study terms with more ambiguous meanings. Lastly, the term “pleasant,” which is considered to have very similar valence and arousal to “happiness,” was added allowing a gauge of whether ratings remained consistent across different emotional terms, where the valence and arousal only slightly change. Table 11 summarizes the responses.

TABLE 11
Mean and Standard Deviation of Emotion Ratings
for Each Chord Progression and Music Background

| | | | Counter- | Limited | Fully |
|---|---|---|---|---|---|
| Emotion | Background | Clockwise | Clockwise | Random | Random |
| Happy | Novice | 4.09 ± 1.78 | 4.82 ± 1.53 | 3.8 ± 1.54 | 2.5 ± 1.43 |
| | Experienced | 4.42 ± 1.29 | 4.54 ± 1.50 | 4.33 ± 0.99 | 3.25 ± 1.30 |
| Pleasant | Novice | 4.18 ± 1.70 | 5.18 ± 1.19 | 4.0 ± 1.61 | 3.4 ± 2.00 |
| | Experienced | 4.67 ± 1.28 | 4.67 ± 1.57 | 3.46 ± 1.41 | 3.29 ± 0.92 |
| Satisfying | Novice | 4.36 ± 1.92 | 5.0 ± 1.54 | 4.3 ± 1.74 | 3.7 ± 2.1 |
| | Experienced | 4.83 ± 1.21 | 4.75 ± 1.51 | 3.83 ± 1.55 | 3.29 ± 1.54 |
| Tiring | Novice | 3.90 ± 1.50 | 2.64 ± 1.07 | 2.7 ± 1.1 | 2.9 ± 1.3 |
| | Experienced | 2.83 ± 1.28 | 3.08 ± 1.32 | 3.33 ± 1.49 | 3.71 ± 1.54 |

The effect of different resolution levels on perception was analyzed in clockwise, counter-clockwise, and limited random conditions. The study found that the counter-clockwise condition was perceived as the happiest among the participants, with a mean of 4.629 and a standard deviation of 1.513. The clockwise condition was the second happiest, with a mean of 4.314 and a standard deviation of 1.469. However, the difference in happiness levels between the clockwise and counter-clockwise conditions was not statistically significant according to the Kruskal-Wallis test (p=0.404). The limited random condition was perceived as sadder than both conditions, with a mean of 4.176 and a standard deviation of 1.200. This condition, however, was not significantly sadder than the clockwise (p=0.510) or counter-clockwise (p=0.125) conditions. These results suggest that progressions with some level of resolution may sound happier to users, although the difference is relatively minor.

Next, the effect of the inclusion of chords beyond major triads on perceived happiness was analyzed. To do this, the limited random was compared to the fully random condition. It was found that the limited random condition sounds significantly happier than the fully random condition (p=0.002). This indicates that limiting chord progressions to major triads can make a song sound happier. However, it is important to note that some changes in perceptions may sound sad due to their random nature being less intuitive for listeners to follow. To investigate this confound, the second study was performed as will be described below.

Next, the relationship between a participants' music education background and how they perceive the harmonies was analyzed. There was no significant difference between how happy participants with and without music education perceived a harmony to be across all four types of progressions according to a Kruskal-Wallis test (Clockwise: p=0.623, Counter-Clockwise: p=0.496, Limited Random: p=0.224, Fully Random: p=0.164). While these tests indicate musical background does not affect how the progressions were perceived, an interesting trend exists within the data. For all progressions other than the counter-clockwise condition, participants with musical experience rated the progression as sounding happier than non-musical participants. This indicates, although not significantly, that participants with less music education were more affected by the weakly resolved chord progressions, perceiving their open-ended resolution positively. It is also possible that participants with musical backgrounds tend to focus less on the resolution of the chord progression, which is also discussed more below with regard to the second study.

To better understand whether music's happiness is perceived differently, the correlation between happiness and satisfaction, pleasantness, and tiredness was analyzed for both novice and experienced participants. According to Pearson R tests, experienced participants' ratings have happiness positively correlated with pleasant (p=2.098×10⁻⁵) and satisfying (p=0.002), and negatively correlated with tiring (p=0.001). Ratings from the novice participants revealed no significant correlation between happiness and satisfaction (p=0.086), or tiredness (p=0.123). There was, however, a positive correlation between pleasantness and happiness (p=0.026) for these participants. These results appear to emphasize that participants with a musical background are more consistent with their perceptions of music emotionality than novice participants.

While the first study identified that altering the resolution of a chord progression may change how happy the harmony is perceived, it also identified several possible confounding variables. The second study strives to provide participants with more freedom to associate harmonies with emotions, better removing these confounds. Specifically, the second study investigates the two findings from the first study that could be the result of confounding variables: RQ1—Are chords other than major triads used to make a harmony sound sadder? RQ2—Do participants with varying music backgrounds use different musical elements to express specific emotions?

These questions were investigated in an interactive session where participants composed chord progression to sound either happy or sad. Eight college-aged participants were recruited for the second study. P2 identified as female, while the other seven identified as male. Recruited participants had varying music backgrounds. P1 and P2 had no music education. P3 and P4 both played musical instruments growing up. P3 played for only a year while P4 played for multiple years. P5 no longer played instruments but remained confident in their ability to read music. P6 and P7 both played instruments at the time of this study and could read music. P7 was highly confident in their musical background, which was consequently entirely self-taught. P8 was an award-winning pianist and a member of local music ensembles. Participants' numbers were assigned such that participants with higher numbers had more musical backgrounds. P1 and P4 were new participants, while the rest were re-recruited from the first study.

The participants were given a task to generate two chord progressions, one that sounded “happy” and another that sounded “sad.” To accomplish this, they utilized a web application called ChordChord, which is displayed in FIG. 21. The tool allows users to intuitively create chord progressions and drum tracks with a variety of options while hearing how every change sounds in real-time. This particular application was chosen due to its user-friendly interface and the diverse range of options it provides. Users can also decide the length of their chord progressions and design a supporting drum track.

Participants were first given a brief tutorial on how to use ChordChord. After learning the software, participants were instructed to create a chord progression that reflected a particular emotion. They then used ChordChord to create a drum track that complemented the harmony. After creating both the chord progression and drum track, participants were asked which of the two influenced the song's valence more. Participants were monitored to ensure they properly followed the procedure.

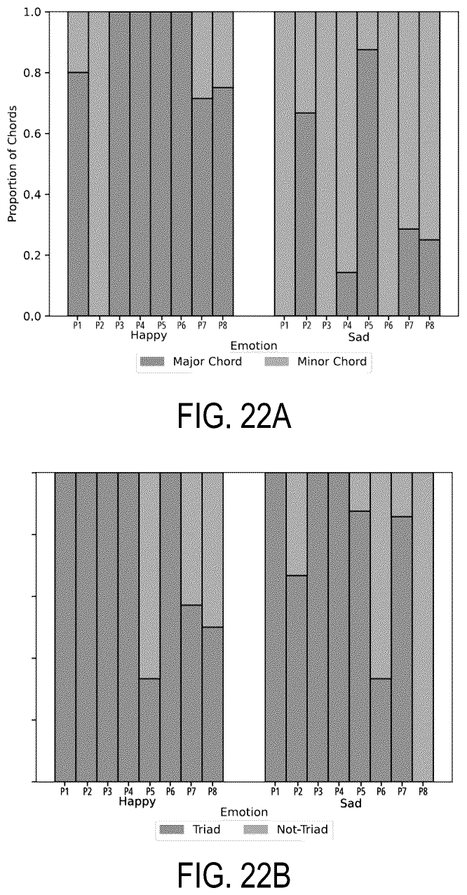

In comparing the chord progressions designed to evoke emotions of happiness and sadness, there was a stark difference in the usage of major and minor chords. 72% of chords used in the “happy” harmonies were in a major key while 70% of the chords used in “sad” harmonies were in a minor key. This difference is not unexpected, as major keys are generally considered to sound happier than minor keys, but it is still important to note. The use of major and minor chords was then analyzed on a per-participant level, as shown in FIG. 22A, which displays the proportion of chords in each participants' designed progression for happy and sad. From FIG. 22A, it can be seen that are differences in how various participants used major and minor chords. Most notably, the participants with the most and least music experience were the only four to use minor chords in a “happy” progression. P2 in particular, only used minor chords to sound “happy.” Participants' “sad” harmonies also used different rates of minor chords. P2 and P5 used more major than minor chords. The two highly musical participants, P7 and P8, used very similar rates of major chords in this sad “melody,” while the other two participants were far more variable. This may indicate that participants with varying levels of music background perceive the “happiness” and “sadness” of major and minor chords differently.

The use of resolved chord progressions (I-V or I-IV) to express emotions, without being instructed to do so was also inspected. Only the highly-resolved transition from root to dominant (I-V) was used in three “happy” progressions. This finding supports the earlier conclusion that resolved progressions sound happier. However, the first study suggested that this transition would be used by less musical participants, which contradicts the findings of the second study. It was observed that P3, P4, and P6, three participants with some musical background, used this transition. The transition from root to subdominant (I-IV) was only used in P7's “happy” progression. This supports the conclusion that resolved chord progressions may sound different based on a participant's musical background, but it does not clarify whether they follow a predictable trend.

Expanding on the first study, the effect of chords other than traditional triads affect on perceived emotional response to a harmony was investigated. The progressions created by participants were analyzed to determine whether they used any chords other than a major or minor triad, such as a tetrad. FIG. 22B shows the usage rates for triads and other types of chords. Interestingly, non-triads were primarily used by participants with some level of music experience. P2, however, served as an exception, using non-triads with no music experience. Non-triads were used more frequently in “sad” harmonies. This further supports the idea that these chords make a song sound sadder. The increased usage by musical participants is also noteworthy, suggesting that these changes may be more noticeable to individuals with more musical training.

Finally, the effect of a participant's musical background on their emotional response to musical elements other than a harmony was investigated. Specifically, participants were asked to identify which musical voice-harmony or another-they found to be more emotional. In the context of the study, participants used drums to provide an additional voice, albeit simplified compared to a traditional melody to ensure their focus during design remained on the chord progression. P1, P2, P4, P5 and P7 believed that the emotional message of a song is influenced more by the chords than the designed drum track. P3 thought that the chords and drums had an equal influence on the conveyed emotion. Overall, these responses suggest that participants with less musical experience tend to find the harmony of a song to be more emotional. However, P6 and P8 disagreed. P8 argued that a drum track with high velocity could make almost any chord progression sound happy, while P6 indicated that he would normally find chords to influence emotions more, but in this specific composition, the drums held a greater influence. These responses contradict the views of less musical participants and suggest that participants with more musical experience may be more likely to focus on different elements within the music to understand the conveyed emotion.

Both studies indicate chord progressions that resolve better sound happier. Additionally, harmony sounds happier when it uses exclusively triads. However, the second provides strong evidence that the most significant difference between happy and sad-sounding harmonies is whether the chords are played in a major or minor key. These studies expand other findings, demonstrating that despite variations, these trends appear to be generally followed regardless of a participants' musical background. Systems can use these results to design harmonies that communicate wellness information while evoking specific emotions.

While the studies showed that perceptions of musical emotions generally abide by the aforementioned trends, slight variations were observed between participants with different musical backgrounds. In the first study, it was observed that participants may perceive harmonies as equally happy or sad, but novice participants may be less consistent with their interpretations. The second study revealed several potential differences in emotional musical preferences. Interestingly, participants with the most and least musical experience used minor chords in their “happy” chord progressions. In contrast, participants with some (but not a lot) of music experience exclusively used major chords in the “happy” progression. This could indicate that participants without music experience are less sensitive to traditional music-emotion rules, and participants with much experience are willing to deviate from these rules. However, participants with some music experience but not much may know the association between major chords and happiness and are unwilling to deviate from it. The second study reveals another, more straightforward trend that participants with higher levels of music experience appear to be more likely to use non-triads to express emotions. These findings indicate that while participants' perceptions of emotional harmonies follow the general trends, there may be minor differences in how participants of varying levels of musical background perceive the emotions of harmonies. To better understand these apparent nuances, one may expand the analysis to highlight how small changes to chords alter the perceived emotions. Analyzing the differences between different types of chords, such as inversions, might help to better communicate emotions.

Thus, in addition to applications in which the methodologies herein are used to explicitly communicate wellness information, the methodologies used herein may also be sued to embed specific emotions in the music. Musical feedback's strength lies within music's unique connection to emotional processing, meaning systems in accordance with the present disclosure may be capable of delivering feedback in more meaningful ways by evoking specific emotions. Such systems could represent wellness feedback directly in the emotionality, building relatively simple models such as playing happy songs when the feedback is positive. This approach may leverage accurate awareness of the user's context and emotional state, meaning controlling the wellness data and emotions separately may result in more accurate models.

### Example Implementations

The systems and methods described in the above experimental analyses may be implemented in a wide range of use cases. For example, the techniques described above may be used to communicate wellness information to a user and/or to alter or address behaviors of the user. In examples, the techniques may be used to ameliorate symptoms of behavioral health conditions, such as depression, anxiety, dementia, and the like. The techniques may implement data pertaining to the user, such as music modeling surveys completed by the user (or by one or more other users), health or wellness information pertaining to the user (e.g., steps or other measures of physical activity), user background (e.g., music training or education), mood, emotional state (e.g., agitation, mood, irritability, etc.), behavioral health conditions, user feedback, and the like. Such data may be obtained from the user or from another individual, such as a family member of health care provider such as a music therapist. Music may be played for any purpose described above using a wide range of delivery media, such as smartphones, smartwatches, smart speakers, in-vehicle entertainment units, or any other device capable of reproducing music.

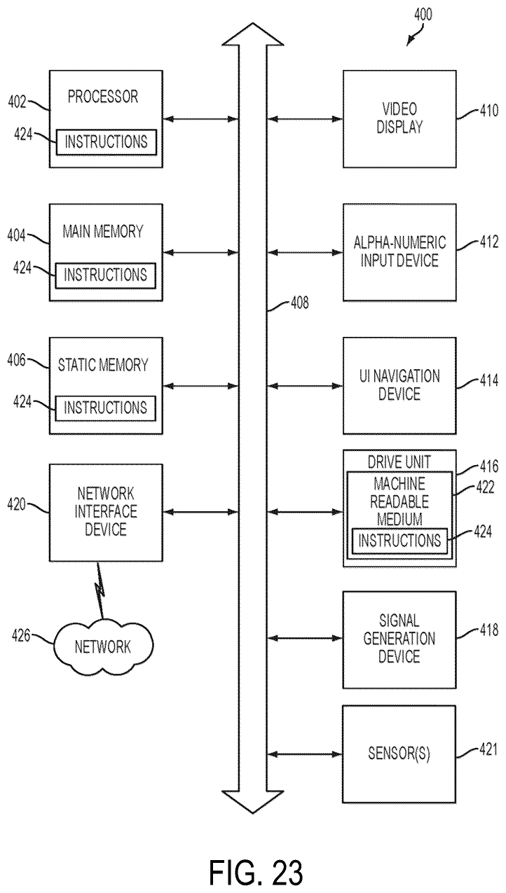

FIG. 23 is a block diagram illustrating an example of a machine upon which one or more aspects of embodiments of the present invention can be implemented. Referring to FIG. 23, an aspect of an embodiment of the present invention includes, but not limited thereto, a system, method, and computer readable medium that provides: a) a musical feedback system converts biobehavioral data collected from personal devices to personalized musical melodies to convey the general health status, b) using sonification to convey users' wellness levels through music, c) using sonification in delivering and communicating health and wellness status on personal devices, d) use of sonification for delivering and communicating personal health information, and e) using personalized sonification models to communicate health information and/or modify behavioral parameters.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, an FPGA, or an ASIC. In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., an FPGA or an ASIC).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines. In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a CPU, a GPU, or both), a main memory 404, and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein, and may be any type of “memory” described above. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., EPROM, EEPROM, and flash memory devices); magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

In examples, the machine 400 may be a personal electronic device associated with the user, such as a smartphone, a smartwatch, another wearable device, or a smart speaker. In the particular case in which the machine 400 is a smartphone, the video display 410, the alpha-numeric input device 412, and the UI navigation device 414 may be implemented as a touchscreen display of the smartphone; the signal generation device 418 may be an audio output of the smartphone (e.g., a Bluetooth transceiver, a headphone jack, an on-device speaker, and the like); and the sensor(s) 421 may be an accelerometer or a microphone of the smartphone.

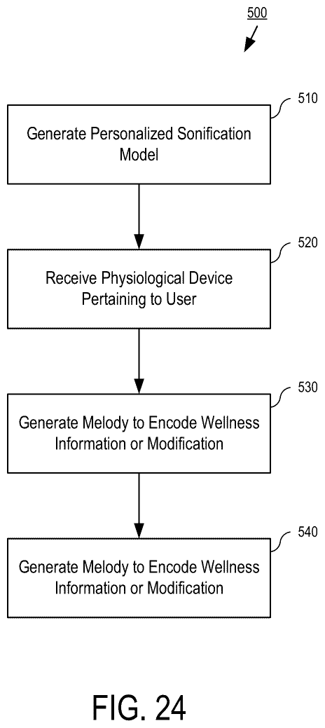

In any implementation, the machine 400 may be configured to perform any one or more of the methods and methodologies set forth herein. FIG. 24 illustrates one example of a method 500 of using personalized sonification models to communicate health information and/or modify wellness (e.g., modify behavior), which may be implemented by generating music that encodes personalized information. For purposes of illustration, the method 500 is described as being performed by the machine 400 (e.g., in the form of the instructions 424 internal to the machine 400 and/or instructions located on an external device for cloud-based or SaaS implementations, which are executed by the processor 402).

The method 500 includes an operation 510 of generating a personalized sonification model, for example based on music modeling data pertaining to a user. The music modeling data may include a personalized relationship parameter, wherein the personalized relationship parameter represents a relationship between a musical feature and a perceived wellness level. The musical feature may be or include at least one of a tempo, a pitch, a key, a dynamic, a smoothness, or a chord progression. Additionally or alternatively, the musical feature may be or include at least one of a slur, an accent, a crescendo, a decrescendo, or a vibrato. Operation 510 may include presenting the user with one or more surveys configured or intended to determine the user's music preferences for each of one or more wellness levels. In example, the surveys may be similar to or the same as those described above with regard to FIGS. 2-15.

The method 500 includes an operation 520 of receiving physiological data pertaining to the user. The physiological data may be related to at least one of a physical wellness or a behavioral wellness of the user. Operation 520 may include receiving sensor data from a device (e.g., a smartphone, a wearable device, etc.) associated with the user. The sensor data may include at least one of a physical activity data (e.g., steps), a mood data, or an emotional state data. The sensor data may be utilized in a raw form, or may be modified or processed prior to utilization. In one example as described above, the sensor data may be converted to one or more scores (e.g., a score for each aspect or parameter in the sensor data), and a physical activity data, mood data, or emotional state data may be determined from the score(s). The physiological data may, at any point in the method 500, be present in the form of raw data and/or encrypted/anonymized data.

The method 500 includes an operation 530 of generating a melody. The melody may encode a wellness information and/or a wellness modification, and may be based on the personalized sonification model and the physiological data (e.g., as described above with regard to operations 510 and 520). In one example, operation 530 may correspond to creating a new melody. In this example, operation 530 may include creating a melody having notes or note combinations configured to encode the wellness information or modification. The encoding may be based on the personalized sonification model; for example, if a user indicates that fast tempo melodies correspond to high wellness, and the method 500 seeks to convey high wellness to the user, then the notes of the melody generated by operation 530 may have a fast tempo. In another example, operation 530 may correspond to modifying a preexisting melody. In this example, operation 530 may include retrieving a preexisting song (e.g., from a playlist established by the user) and modifying at least one musical feature of the preexisting song to encode the wellness information or modification. As above, the modification may be based on the personalized sonification model; for example, if a user indicates that fast tempo melodies correspond to high wellness, and the method 500 seeks to convey high wellness to the user, then operation 530 may increase the tempo of the preexisting song.

The method 500 includes an operation 540 of providing the melody to the user, thereby to convey the wellness information or modification. Operation 540 may include outputting an audio signal corresponding to the melody via an audio output of the user's personal electronic device (e.g., a smartphone), transmitting the audio signal to another device in a wired or wireless manner, playing the melody to the user via the user's personal electronic device (e.g., a smartphone, an alarm clock, etc.), emailing the melody to the user, transmitting the melody to the user via a multimedia messaging service (MMS) or other communication protocol, and the like. In some examples, after the melody has been provided to the user, the method 500 may further include receiving a feedback from the user regarding the provided melody and modifying the personalized sonification model based on the feedback (e.g., for future iterations of the method 500).

Various operations of method 500 may be performed locally or remotely (e.g., cloud-based or SaaS). In remote implementations, a music generation model or program may be stored on a server, and the user's local device may be configured to transmit data to the server (e.g., the physiological data) and receive the melody from the server. Such implementations may be useful where the music generation model or program is processing resource-intensive. In other implementations (e.g., where the user's local device has sufficient processing power), the melody may be generated locally.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims) including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A method of generating music that encodes personalized information, the method comprising:
generating a personalized sonification model based on music modeling data pertaining to a user;
receiving physiological data pertaining to the user, wherein the physiological data is related to at least one of a physical wellness or a behavioral wellness of the user;
generating a melody, wherein the melody encodes a wellness information or modification based on the personalized sonification model and the physiological data; and
providing the melody to the user to convey the wellness information or modification.

2. The method of claim 1, wherein the music modeling data includes a personalized relationship parameter, wherein the personalized relationship parameter represents a relationship between a musical feature and a perceived wellness level.

3. The method of claim 2, wherein the musical feature includes at least one of a tempo, a pitch, a key, a dynamic, a smoothness, or a chord progression.

4. The method of claim 1, wherein receiving the physiological data includes receiving sensor data from a smartphone or wearable device associated with the user.

5. The method of claim 4, wherein the sensor data includes at least one of physical activity data, mood data, or emotional state data.

6. The method of claim 4, wherein receiving the physiological data generating a score corresponding to the sensor data and converting the score to a physical activity level.

7. The method of claim 1, wherein generating the melody includes creating the melody with notes or note combinations configured to encode the wellness information or modification.

8. The method of claim 1, wherein generating the melody includes retrieving a preexisting song, and modifying at least one musical feature of the preexisting song to encode the wellness information or modification.

9. The method of claim 1, further comprising:
receiving a feedback from the user regarding the provided melody; and
modifying the personalized sonification model based on the feedback.

10. A non-transitory computer-readable medium storing instructions that, when executed by at least one processor of a computing device, cause the computing device to perform operations comprising the method of claim 1.

11. A system for generating music that encodes personalized information, the system comprising:
at least one processor; and
a memory operatively connected to the at least one processor and storing instructions that, when executed by the at least one processor, cause the system to perform operations comprising:
generating a personalized sonification model based on music modeling data pertaining to a user,
receiving physiological data pertaining to the user, wherein the physiological data is related to at least one of a physical wellness or a behavioral wellness of the user,
generating a melody, wherein the melody encodes a wellness information or modification based on the personalized sonification model and the physiological data, and
providing the melody to the user to convey the wellness information or modification.

12. The system of claim 11, wherein the music modeling data includes a personalized relationship parameter, wherein the personalized relationship parameter represents a relationship between a musical feature and a perceived wellness level.

13. The system of claim 12, wherein the musical feature includes at least one of a tempo, a pitch, a key, a dynamic, a smoothness, or a chord progression.

14. The system of claim 11, wherein receiving the physiological data includes receiving sensor data from a smartphone or wearable device associated with the user.

15. The system of claim 14, wherein the sensor data includes at least one of physical activity data, mood data, or emotional state data.

16. The system of claim 11, wherein generating the melody includes creating the melody with notes or note combinations configured to encode the wellness information or modification.

17. The system of claim 11, wherein generating the melody includes retrieving a preexisting song, and modifying at least one musical feature of the preexisting song to encode the wellness information or modification.

18. The system of claim 11, wherein the system is a smartphone or wearable device associated with the user.

19. The system of claim 18, wherein providing the melody to the user includes outputting an audio signal corresponding to the melody via an audio output of the smartphone or wearable device.

20. The system of claim 18, wherein generating the melody includes transmitting the physiological data to an external device, and receiving the melody from the external device.
